(19) United States
(12) Patent Application Publication
(10) Pub. No.: US 2024/0124424 A1
(43) Pub. Date: Apr. 18, 2024

(54) SOLID FORMS OF (5S)-CYCLOPROPYL-5-[3-[(3S)-4-(3,5-DIFLUOROPHENYL)-3-METHYL-PIPERAZIN-1-YL]-3-OXO-PROPYL]IMIDAZOLIDINE-2,4-DIONE

(71) Applicant: Galapagos NV, Mechelen (BE)

(72) Inventors: Renaud Henri Marcel LÉPINE, Paris (FR); Didier Philippe Robert SCHILS, Mechelen (BE); Sam Bob CORVELEYN, Mechelen (BE); Michael Anthony LYNCH, Suresnes (FR); Nicolas Valentin LEBLANC, Suresnes (FR); Gradus Johannes DULOS, Leiden (NL)

(21) Appl. No.: 18/267,395

(22) PCT Filed: Dec. 13, 2021
(86) PCT No.: PCT/EP2021/085376

§ 371 Date: Jun. 14, 2023

(30) Foreign Application Priority Data
Dec. 15, 2020 (EP) 20306578.4
Apr. 26, 2021 (EP) 21170505.8

(51) Int. Cl. C07D 403/06; A61P 13/12; A61P 17/00; A61P 19/00; A61P 21/00

(52) U.S. Cl. CPC C07D 403/06; A61P 13/12; A61P 17/00; A61P 19/00; A61P 21/00

(57) ABSTRACT

The present invention relates to solid forms of (5S)-cyclopropyl-5-[3-[(3S)-4-(3,5-difluorophenyl)-3-methyl-piperazin-1-yl]-3 -oxo-propyl]imidazolidine-2,4-dione and to pharmaceutical preparations comprising them and methods of their manufacture, as well as the use of said solid forms or preparations for the prophylaxis and/or treatment of inflammatory conditions, muscular disease, fibrotic diseases, viral infection, and/or diseases involving degradation of cartilage and/or disruption of cartilage homeostasis, especially osteoarthritis.

### FIELD OF THE INVENTION

Herein are provided inter alia, solid forms of the invention and to pharmaceutical compositions comprising them and methods of their manufacture, as well as the use of said solid forms or compositions for the prophylaxis and/or treatment of inflammatory conditions, muscular disease, fibrotic diseases, viral infection, and/or diseases involving degradation of cartilage and/or disruption of cartilage homeostasis.

### BACKGROUND OF THE INVENTION

ADAMTS-5 was identified in 1999 (Abbaszade et al., 1999). In 2005, two independent groups identified ADAMTS-5 as the principal aggrecanase in mouse cartilage (Glasson et al., 2005; Stanton et al., 2005). Proteolysis of aggrecan by ADAMTS-5 occurs at different sites: however cleavage at the Glu373-Ala374 bond (aggrecan IGD) is likely more important in the pathogenesis of osteoarthritis and inflammatory arthritis since a loss of integrity at this bond results in the loss of an entire aggrecan molecule, which is highly detrimental to cartilage integrity and function (Little et al., 2007).

Studies in genetically engineered mouse models (GeMMs) have demonstrated that ADAMTS-5 ablation protects against cartilage damage and aggrecan loss after osteoarthritis induction through surgical instability of the medial meniscus (DMM) (Glasson et al., 2005). Moreover in the DMM model ADAMTS-5 knock-out mice showed reduced subchondral bone changes (Hotter et al., 2009) and did not develop osteoarthritis-associated mechanical allodynia (Malfait et al., 2010). Besides preclinical evidence, clinical evidence also indicates the importance of and interest in ADAMTS-5 as a target for osteoarthritis. Recently, studies with an antibody targeting ADAMTS-5 (Chiusaroli et al., 2013) have been reported. ELISA' s have been developed allowing the measurement of aggrecanase-derived cartilage neo-epitope levels in the synovial fluid as well as blood from rodents to human. This method revealed increased levels of ADAMTS-5 derived neo-epitope levels in the joints of rats in which cartilage degradation was induced by meniscal tear as well as in joints of osteoarthritis patients, thereby providing further translational evidence for the importance of this protease in the development of osteoarthritis (Chockalingam et al., 2011; Larsson et al., 2014).

These findings provide strong evidence for a central role of ADAMTS-5 in osteoarthritis pathology as a key target and an ADAMTS-5 inhibitor capable to reach the joint cartilage at sufficient levels is expected to exert a protective effect on cartilage in osteoarthritic patients.

More recently, the role of ADAMTS5 in has been established in further diseases including muscular disease (Addinsall et al., 2020), liver fibrosis (Bauters et al., 2018, 2016), kidney fibrosis (Collins and Wann, 2020; Taylor et al., 2020), lung fibrosis including IPF (Pardo et al., 2008), and/or viral infections including influenza (McMahon et al., 2016).

In this context, new drugs are being developed, in particular the compound according to formula (I) (5S)-cyclopropyl-5-[3-[(3S)-4-(3,5-difluorophenyl)-3-methyl-piperazin-1-yl]-3-oxo-propyl]imidazole dine-2,4-dione or GLPG1972 or S201086 or aldumastat:

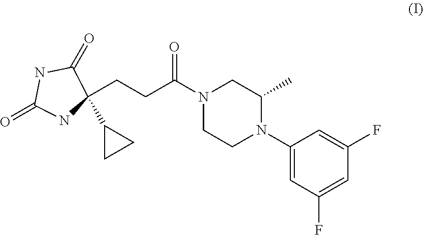 [SMILES: C[C@H]1CN(C(=O)CC[C@@]2(C3CC3)NC(=O)NC2=O)CCN1c1cc(F)cc(F)c1]

(5S)-cyclopropyl-5-[3-[(3S)-4-(3,5-difluorophenyl)-3-methyl-piperazin-1-yl]-3-oxo-propyl] imidazolidine-2,4-dione of formula (I) is disclosed in WO 2016/102347 and is an ADAMTS-5 inhibitor which in turn may be useful in the prophylaxis and/or treatment of inflammatory conditions, muscular disease, fibrotic diseases, viral infection, and/or diseases involving degradation of cartilage and/or disruption of cartilage homeostasis, especially osteoarthritis.

An important characteristic of various bioactive substances (for example but without limitation, pharmaceuticals, medicines and biocides, usually referred to as drugs) is their “bio-availability” or active concentration in a form which can be absorbed and utilized by a target organ or organism. In many cases, bioavailability is related to drug solubility in water, which may depend on many parameters such as acid-basic properties, and/or polymorphism.

Drugs in their free base form may be poorly soluble in water, but the presence of acidic sites (for example carboxylic acids, phenols, sulfonic acids) or basic sites (for example amino groups, basic nitrogen centres) can be used advantageously to produce salts of the drug. The resulting ionic compounds become much more soluble in water by virtue of their ionic character and lower dissolution energy, and thus may improve bioavailability. A guideline of 50 μg/mL for aqueous solubility is provided by Lipinski et al. (Lipinski et al., 2001)

Salt forming agents are available in large number, and salt selection must be carefully designed. The aim of the salt selection is to identify the best salt form suitable for development, and is based primarily on four main criteria: aqueous solubility at various pH, high degree of crystallinity, low hygroscopicity, and optimal chemical stability. (Stahl et al., 2011)

Polymorphism is a solid-state property of some molecules (and molecular complexes) wherein a single molecule may give rise to a variety of distinct crystal structures with different physical properties which may be characterized by determining melting point, thermal behaviors using thermogravimetric analysis (TGA), or differential scanning calorimetry (DSC), X-ray pattern diffraction (XRPD), infrared absorption fingerprint, and/or solid state (¹³C) NMR spectrum.

If a suitable solid form, such as a crystalline or polymorphic form of a drug or salt thereof can be identified, further investigations can be performed to identify alternative solid forms both qualitatively and quantitatively. The availability of such solid forms is highly unpredictable and can require a combination of intuition, careful empirical design, perseverance, and serendipity. On top of the challenges associated with even finding one or more defined solid forms, the properties of any forms thus discovered need to be carefully evaluated to see if one or more of them is actually suitable for pharmaceutical development. Indeed, in a first aspect, crystallinity of drug can affect, among other physical and mechanical properties, solubility, dissolution rate, flowability, hardness, compressibility, and/or melting point. In a second aspect, a crystalline form may have advantages over the amorphous form, for example, purification to the high degree of purity required by most regulatory authorities could be more efficient and therefore cost less for the crystalline form than for the amorphous solid. In addition, handling of the crystalline form could be improved over the amorphous form, which could be oily, or sticky for example, and in practice, drying of a crystalline material which has a well-defined drying or desolvation temperature could in some cases be more easily controlled, than for the amorphous solid which could have a greater affinity for organic solvents and variable drying temperature. Finally, downstream processing of the crystalline drug can in some cases permit enhanced process control. In a third aspect, physical and chemical stability, and/or shelf-life could be improved for crystalline forms over amorphous forms.

Further pharmacokinetic and pharmacodynamic properties of a drug could be linked to a particular solid or crystalline structural form, and it could be paramount to produce and retain the same form from production to administration to the patient. Therefore the obtention of salts, and/or crystalline forms over amorphous materials is highly desirable.(Hilfiker et al., 2006)

Because drug compounds having, for example, improved stability, solubility, shelf life and in vivo pharmacology, are consistently sought, there is an ongoing need for new or purer solid forms of existing drug molecules.

### SUMMARY OF THE INVENTION

Herein are provided inter alia, solid forms of the invention and pharmaceutical compositions comprising them and methods of their manufacture, as well as the use of said solid forms or compositions for the prophylaxis and/or treatment of inflammatory conditions, muscular disease, fibrotic diseases, viral infection, and/or diseases involving degradation of cartilage and/or disruption of cartilage homeostasis, in particular osteoarthritis.

Accordingly, in a first aspect, are provided inter alia, solid forms of the invention having a Formula (I)—hereafter Cpd 1: (5S)-cyclopropyl-5-[3-[(3S)-4-(3,5-difluorophenyl)-3-methyl-piperazin-1-yl]-3-oxo-propyl]imidazolidine-2,4-dione of formula (I):

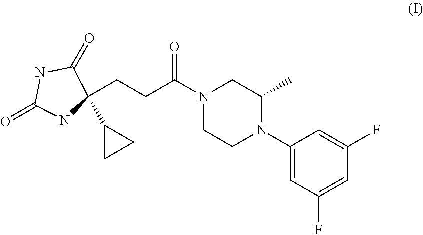 [SMILES: C[C@H]1CN(C(=O)CC[C@@]2(C3CC3)NC(=O)NC2=O)CCN1c1cc(F)cc(F)c1]

In a first aspect, a solid form of the invention is a crystalline form. In a more particular aspect, the solid form of the invention is anhydrous. In another aspect, the solid form is hydrated. In another aspect, the solid form is solvated.

In another aspect, a solid form of the invention is amorphous. In another aspect, the solid form is solvated. In another aspect, the solid form is unsolvated.

In a particular aspect, solid forms of the invention are provided inter alia for use in the prophylaxis and/or treatment of inflammatory conditions, muscular disease, fibrotic diseases, viral infection, and/or diseases involving degradation of cartilage and/or disruption of cartilage homeostasis.

Furthermore, it has also been unexpectedly demonstrated that a solid form of the invention exhibits improved exposure compared to other solid-state forms of Cpd 1.

In a further aspect, are provided inter alia, pharmaceutical compositions comprising a solid form of the invention, and a pharmaceutical carrier, excipient or diluent. In a particular aspect, the pharmaceutical composition may additionally comprise further therapeutically active ingredients suitable for use in combination with the solid form of the invention. In a more particular aspect, the further therapeutically active ingredient is an agent for the prophylaxis and/or treatment of inflammatory conditions, muscular disease, fibrotic diseases, viral infection, and/or diseases involving degradation of cartilage and/or disruption of cartilage homeostasis.

Moreover, the solid forms of the invention, useful in the pharmaceutical compositions and treatment methods provided herein, are pharmaceutically acceptable as prepared and used.

In a further aspect, is provided inter alia, a method of treating a mammal, in particular humans, afflicted with a condition selected from among those listed herein, and particularly inflammatory conditions, muscular disease, fibrotic diseases, viral infection, and/or diseases involving degradation of cartilage and/or disruption of cartilage homeostasis, which method comprises administering an effective amount of the pharmaceutical composition or solid form of the invention as described herein.

Herein are also provided inter alia, pharmaceutical compositions comprising a solid form of the invention, and a suitable pharmaceutical carrier, excipient or diluent for use in medicine. In a particular aspect, the pharmaceutical composition is for use in the prophylaxis and/or treatment of inflammatory conditions, muscular disease, fibrotic diseases, viral infection, and/or diseases involving degradation of cartilage and/or disruption of cartilage homeostasis.

In additional aspects, herein are provided inter alia, methods for synthesizing the solid forms of the invention, with representative synthetic protocols and pathways disclosed later on herein.

Other objects and advantages will become apparent to those skilled in the art from a consideration of the ensuing detailed description.

### DESCRIPTION OF THE FIGURES

FIG. 1: X-ray powder diffraction profile of crystalline Form I

FIG. 2: X-ray powder diffraction profile of crystalline Form II

FIG. 3: DSC profile of crystalline Form I

FIG. 4: DSC profile of crystalline Form II

FIG. 5: TGA profile of crystalline Form I

FIG. 6: TGA profile of crystalline Form II

FIG. 7: IR spectrum of crystalline Form I

FIG. 8: IR spectrum of crystalline Form II

FIG. 9: ¹³C solid state NMR spectrum of crystalline Form I

FIG. 10: ¹³C solid state NMR spectrum of crystalline Form II

FIG. 11: ¹⁵N solid state NMR spectrum of crystalline Form I

FIG. 12: ¹⁵N solid state NMR spectrum of crystalline Form II

FIG. 13: DVS profile of crystalline Form I

FIG. 14: DVS profile of crystalline Form II

FIG. 15: X-ray powder diffraction profile of crystalline Form III

FIG. 16: DSC profile of crystalline Form III

FIG. 17: TGA profile of crystalline Form III

FIG. 18: X-ray powder diffraction profile of amorphous Cpd 1

FIG. 19: TGA profile of amorphous Cpd 1

FIG. 20: DSC profile of amorphous Cpd 1

FIG. 21: shows the urine ratio protein/creatinine for Cpd 1 (Group A, filled circles), vehicle (Group B, filled squares), lisinopril (Group C, filled upward triangles), and sham (Group D, filled downward triangles)

FIG. 22: shows the muscle grip strength corrected for body weight (g/g)—y axis—in the Duchenne dystrophy model (mice mdx assay) at the 3 time points pre-treatment, mid treatment, and end of treatment—x-axis—for each groups vehicle (filled circles), Cpd 1 (filled squares), prednisolone (filled upwards triangles), and Cpd 1+prednisolone (filled downwards triangles)

FIG. 23: shows the bone volume fraction (bone volume/tissue volume, %, y axis) in the Duchenne dystrophy model (mice mdx assay) after treatment for the vehicle group (A), for the Cpd 1 group (B), for the prednisolone group (C), and for the combination prednisolone+Cpd 1 group (D) (x axis)

### DETAILED DESCRIPTION OF THE INVENTION

### Definitions

The following terms are intended to have the meanings presented therewith below and are useful in understanding the description and intended scope of the present invention.

When describing the invention, which may include compounds, pharmaceutical compositions containing such compounds and methods of using such compounds and compositions, the following terms, if present, have the following meanings unless otherwise indicated. It should also be understood that when described herein any of the moieties defined forth below may be substituted with a variety of substituents, and that the respective definitions are intended to include such substituted moieties within their scope as set out below. Unless otherwise stated, the term “substituted” is to be defined as set out below. It should be further understood that the terms “groups” and “radicals” can be considered interchangeable when used herein.

The articles ‘a’ and ‘an’ may be used herein to refer to one or to more than one (i.e. at least one) of the grammatical objects of the article. By way of example ‘an analogue’ means one analogue or more than one analogue.

‘Pharmaceutically acceptable’ means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

‘Pharmaceutically acceptable salt’ refers to a salt of a compound of the invention that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g. an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non-toxic organic or inorganic acids, such as hydrochloride, hydrobromide, tartrate, mesylate, acetate, maleate, oxalate and the like. The term ‘pharmaceutically acceptable cation’ refers to an acceptable cationic counter-ion of an acidic functional group. Such cations are exemplified by sodium, potassium, calcium, magnesium, ammonium, tetraalkylammonium cations, and the like.

‘Pharmaceutically acceptable vehicle’ refers to a diluent, adjuvant, excipient or carrier with which a compound of the invention is administered.

The terms ‘inert solid diluent’ or ‘solid diluent’ or ‘diluents’ refer to materials used to produce appropriate dosage form size, performance and processing properties for tablets and/or capsules. An inert solid diluent can be also referred to as filler or filler material. Particular examples of diluents include cellulose powdered, silicified microcrystalline cellulose acetate, compressible sugar, confectioner's sugar, corn starch and pregelatinized starch, dextrates, dextrin, dextrose, erythritol, ethylcellulose, fructose, fumaric acid, glyceryl palmitostearate, inhalation lactose, isomalt, kaolin, lactitol, lactose anhydrous, lactose monohydrate, and corn starch, spray dried monohydrate and microcrystalline cellulose, maltodextrin, maltose, mannitol, medium-chain triglycerides, microcrystalline cellulose, polydextrose, polymethacrylates, simethicone, sorbitol, pregelatinized starch, sterilizable maize, sucrose, sugar spheres, sulfobutylether β-cyclodextrin, talc, tragacanth, trehalose, or xylitol. More particular examples of diluents include cellulose powdered, silicified microcrystalline cellulose acetate, compressible sugar, corn starch and pregelatinized starch, dextrose, fructose, glyceryl palmitostearate, anhydrous, monohydrate and corn starch, spray dried monohydrate and microcrystalline cellulose, maltodextrin, maltose, mannitol, medium chain triglycerides, microcrystalline cellulose, polydextrose, sorbitol, starch, pregelatinized, sucrose, sugar spheres, trehalose, or xylitol.

‘Lubricant’ refers to materials that prevent ingredients from clumping together and from sticking to the tablet punches or capsule filling machine. Lubricants also ensure that tablet formation and ejection can occur with low friction between the solid and die wall. Particular examples of lubricants include canola oil, hydrogenated castor oil, cottonseed oil, glyceryl behenate, glyceryl monostearate, glyceryl palmitostearate, magnesium stearate, medium-chain triglycerides, mineral oil, light mineral oil, octyldodecanol, poloxamer, polyethylene glycol, polyoxyethylene stearates, polyvinyl alcohol, starch, or hydrogenated vegetable oil. More particular examples of lubricants include magnesium stearate, glyceryl behenate, glyceryl monostearate, or hydrogenated vegetable oil.

‘Disintegrant’ refers to material that dissolve when wet causing the tablet to break apart in the digestive tract, releasing the active ingredients for absorption. They ensure that when the tablet is in contact with water, it rapidly breaks down into smaller fragments, facilitating dissolution. Particular examples of disintegrants include alginic acid, powdered cellulose, chitosan, colloidal silicon dioxide, corn starch and pregelatinized starch, crospovidone, glycine, guar gum, low-substituted hydroxypropyl cellulose, methylcellulose, microcrystalline cellulose, croscarmellose sodium or povidone.

The term ‘colorant’ describes an agent that imparts color to a formulation. Particular examples of colorants include iron oxide, or synthetic organic dyes (US Food and Drug administration, Code of Federal Regulations, Title 21 CFR Part73, Subpart B).

The term ‘plasticizing agent’ or ‘plasticizer’ refers to an agent that is added to promote flexibility of films or coatings. Particular examples of plasticizing agent include polyethylene glycols or propylene glycol.

The term ‘pigment’ refers to an insoluble colouring agent.

The term ‘film-coating agent’ or ‘coating agent’ or ‘coating material’ refers to an agent that is used to produce a cosmetic or functional layer on the outer surface of a dosage form. Particular examples of film-coating agent include glucose syrup, maltodextrin, alginates, or carrageenan.

‘Glidant’ refers to materials that are used to promote powder flow by reducing interparticle friction and cohesion. These are used in combination with lubricants as they have no ability to reduce diewall friction. Particular examples of glidants include powdered cellulose, colloidal silicon dioxide, hydrophobic colloidal silica, silicon dioxide, or talc. More particular examples of glidants include colloidal silicon dioxide, hydrophobic colloidal silica, silicon dioxide, or talc.

‘Flavouring agents’ refers to material that can be used to mask unpleasant tasting active ingredients and improve the acceptance that the patient will complete a course of medication. Flavourings may be natural (e.g. fruit extract) or artificial. Non-limiting examples of flavouring agents include mint, cherry, anise, peach, apricot, liquorice, raspberry, or vanilla.

‘Prodrugs’ refers to compounds, including derivatives of the compounds of the invention, which have cleavable groups and become by solvolysis or under physiological conditions the compounds of the invention which are pharmaceutically active in vivo. Such examples include, but are not limited to, choline ester derivatives and the like, N-alkylmorpholine esters and the like.

‘Solvate’ refers to forms of the compound that are associated with a solvent, usually by a solvolysis reaction. This physical association includes hydrogen bonding. Conventional solvents include water, EtOH, acetic acid and the like. The compounds of the invention may be prepared e.g. in crystalline form and may be solvated or hydrated. Suitable solvates include pharmaceutically acceptable solvates, such as hydrates, and further include both stoichiometric solvates and non-stoichiometric solvates. In certain instances, the solvate will be capable of isolation, for example when one or more solvent molecules are incorporated in the crystal lattice of the crystalline solid. ‘Solvate’ encompasses both solution-phase and isolable solvates. Representative solvates include hydrates, ethanolates and methanolates.

As used herein, ‘solid form(s) of the invention’, and equivalent expressions, are meant to embrace compounds of the Formula(e) as herein described, amorphous or crystalline, which expression includes the pharmaceutically acceptable salts, and the solvates, e.g. hydrates, and the solvates of the pharmaceutically acceptable salts where the context so permits Similarly, reference to intermediates, whether or not they themselves are claimed, is meant to embrace their salts, and solvates, where the context so permits.

As used herein, the term “polymorphs” or “polymorphic forms” refers to crystal forms of the same molecule. Different polymorphic forms of a molecule have different physical properties as a result of the arrangement or conformation of the molecules in the crystal lattice. Some of the different crystal properties include melting temperature, heat of fusion, solubility, dissolution rate and/or vibrational spectra. The physical form of a particular compound is particularly important when the compound is used in a pharmaceutical formulation because different solid forms of a compound result in different properties of the drug product.

Polymorphs of a molecule can be obtained by a number of methods, as shown in the art, such as, for example, melt recrystallization, melt cooling, solvent recrystallization, desolvation, rapid evaporation, rapid cooling, slow cooling, vapor diffusion and sublimation. Techniques for characterizing a polymorph include X-ray powder diffraction (XRPD), single crystal X-ray diffraction (XRD), differential scanning calorimetry (DSC), vibrational spectroscopy (e.g., IR and Raman spectroscopy), solid state nuclear magnetic resonance (ssNMR), hot stage optical microscopy, scanning electron microscopy (SEM), electron crystallography and quantitative analysis, particle size analysis (PSA), surface area analysis, solubility studies and dissolution studies.

The term “hydrate” refers to the chemical entity formed by the interaction of water and a compound.

As used herein, the term “dihydrate” refers a hydrate that contains two molecules of water per one molecule of the substrate.

As used herein, the term “crystalline” refers to a solid in which the constituent atoms, molecules or ions are arranged in a regularly ordered, repeating pattern in three dimensions.

The specification and claims contain listing of species using the language “selected from . . . and . . . ” and “is . . . or . . . ”. When this language is used in this application, unless otherwise stated it is meant to include the group as a whole, or any single members thereof, or any subgroups thereof. The use of this language is merely for shorthand purposes and is not meant in any way to limit the removal of individual elements or subgroups as needed.

When ranges are referred to herein, the citation of a range should be considered a representation of each member of said range.

‘Subject’ includes humans. The terms ‘human’, ‘patient’ and ‘subject’ are used interchangeably herein.

‘Effective amount’ means the amount of a compound of the invention that, when administered to a subject for treating a disease, is sufficient to effect such treatment for the disease. The “effective amount” can vary depending on the compound, the disease and its severity, and the age, weight, etc., of the subject to be treated.

‘Preventing’ or ‘prevention’ refers to a reduction in risk of acquiring or developing a disease or disorder (i.e. causing at least one of the clinical symptoms of the disease not to develop in a subject that may be exposed to a disease-causing agent, or predisposed to the disease in advance of disease onset.

The term ‘prophylaxis’ is related to ‘prevention’, and refers to a measure or procedure the purpose of which is to prevent, rather than to treat or cure a disease. Non-limiting examples of prophylactic measures may include the administration of vaccines; the administration of low molecular weight heparin to hospital patients at risk for thrombosis due, for example, to immobilization; and the administration of an anti-malarial agent such as chloroquine, in advance of a visit to a geographical region where malaria is endemic or the risk of contracting malaria is high.

‘Treating’ or ‘treatment’ of any disease or disorder refers, in one embodiment, to ameliorating the disease or disorder (i.e. arresting the disease or reducing the manifestation, extent or severity of at least one of the clinical symptoms thereof). In another embodiment ‘treating’ or ‘treatment’ refers to ameliorating at least one physical parameter, which may not be discernible by the subject. In yet another embodiment, ‘treating’ or ‘treatment’ refers to modulating the disease or disorder, either physically, (e.g. stabilization of a discernible symptom), physiologically, (e.g. stabilization of a physical parameter), or both. In a further embodiment, “treating” or “treatment” relates to slowing the progression of the disease.

As used herein the term ‘inflammatory diseases’ refers to the group of conditions including rheumatoid arthritis, osteoarthritis, juvenile idiopathic arthritis, psoriasis, psoriatic arthritis, allergic airway disease (e.g. asthma, rhinitis), chronic obstructive pulmonary disease (COPD), inflammatory bowel diseases (e.g. Crohn's disease, ulcerative colitis), endotoxin-driven disease states (e.g. complications after bypass surgery or chronic endotoxin states contributing to e.g. chronic cardiac failure), and related diseases involving cartilage, such as that of the joints. Particularly the term refers to rheumatoid arthritis, osteoarthritis, allergic airway disease (e.g. asthma), chronic obstructive pulmonary disease (COPD) and inflammatory bowel diseases. More particularly the term refers to rheumatoid arthritis, and osteoarthritis (OA). Most particularly the term refers to osteoarthritis (OA).

As used herein the term ‘muscular diseases’ refers to the group of diseases that cause progressive weakness and loss of muscle mass, wherein abnormal genes (mutations) interfere with the production of proteins needed to form healthy muscle. In particular, the term refers to muscular dystrophy. More particular, the term refers to Duchenne type muscular dystrophy, Becker muscular dystrophy, myotonic dystrophy, facioscapulohumeral dystrophy, congenital dystrophy, and/or limb-girdle dystrophy. Most particular, the term refers to Duchenne type muscular dystrophy.

As used herein the term ‘fibrotic diseases’ refers to diseases characterized by excessive scarring due to excessive production, deposition, and contraction of extracellular matrix, and are that are associated with the abnormal accumulation of cells and/or fibronectin and/or collagen and/or increased fibroblast recruitment. In particular, the term refers to fibrosis of individual organs or tissues such as the heart, kidney, liver, joints, lung, pleural tissue, peritoneal tissue, skin, cornea, retina, musculoskeletal and digestive tract. In particular, the term fibrotic diseases refers to pulmonary fibrosis (such as idiopathic pulmonary fibrosis (IPF), progressive fibrosing form of interstitial lung disease (PF-ILD), progressive massive fibrosis (PMF), and/or cystic fibrosis (CF)), other diffuse parenchymal lung diseases of different etiologies including iatrogenic drug-induced fibrosis, occupational and/or environmental induced fibrosis, granulomatous diseases (sarcoidosis, hypersensitivity pneumonia), collagen vascular disease, alveolar proteinosis, langerhans cell granulomatosis, lymphangioleiomyomatosis, inherited diseases (Hermansky-Pudlak Syndrome, tuberous sclerosis, neurofibromatosis, metabolic storage disorders, familial interstitial lung disease); radiation induced fibrosis; chronic obstructive pulmonary disease (COPD); scleroderma; bleomycin induced pulmonary fibrosis; chronic asthma; silicosis; asbestos induced pulmonary fibrosis; acute respiratory distress syndrome (ARDS); kidney fibrosis; polycystic disease (PKD), autosomal dominant polycystic kidney disease (ADPKD), tubulointerstitium fibrosis; glomerular nephritis; focal segmental glomerular sclerosis; IgA nephropathy, membranous nephropathy; hypertension; Alport; gut fibrosis; liver fibrosis; cirrhosis; alcohol induced liver fibrosis; toxic/drug induced liver fibrosis; hemochromatosis; nonalcoholic steatohepatitis (NASH); biliary duct injury; primary biliary cirrhosis; infection induced liver fibrosis; viral induced liver fibrosis; and autoimmune hepatitis; corneal scarring; hypertrophic scarring; Dupuytren disease, keloids, cutaneous fibrosis; cutaneous scleroderma; systemic sclerosis, spinal cord injury/fibrosis; myelofibrosis; vascular restenosis; atherosclerosis; arteriosclerosis; Wegener's granulomatosis; Peyronie's disease, or chronic lymphocytic. More particularly, the term refers to idiopathic pulmonary fibrosis (IPF), progressive fibrosing form of interstitial lung disease (PF-ILD), IgA nephropathy, membranous nephropathy, focal segmental glomerulo sclerosis, autosomal dominant polycystic kidney disease (ADPKD), and/or nonalcoholic steatohepatitis (NASH).

As used herein the term ‘viral infection’ refers to infections in the body caused by a virus. In particular, the term refers to influenza (the flu).

As used herein the term ‘diseases involving degradation of cartilage and/or disruption of cartilage homeostasis’ includes conditions such as osteoarthritis, psoriatic arthritis, juvenile rheumatoid arthritis, gouty arthritis, septic or infectious arthritis, reactive arthritis, reflex sympathetic dystrophy, algodystrophy, achondroplasia, Paget's disease, Tietze syndrome or costal chondritis, fibromyalgia, osteochondritis, neurogenic or neuropathic arthritis, arthropathy, sarcoidosis, amylosis, hydarthrosis, periodical disease, rheumatoid spondylitis, endemic forms of arthritis like osteoarthritis deformans endemica, Mseleni disease and Handigodu disease; degeneration resulting from fibromyalgia, systemic lupus erythematosus, scleroderma and ankylosing spondylitis. More particularly, the term refers to osteoarthritis (OA).

‘Compound(s) of the invention’, and equivalent expressions, are meant to embrace compounds of the Formula(e) as herein described, which expression includes the pharmaceutically acceptable salts, and the solvates, e.g. hydrates, and the solvates of the pharmaceutically acceptable salts where the context so permits. Similarly, reference to intermediates, whether or not they themselves are claimed, is meant to embrace their salts, and solvates, where the context so permits.

When ranges are referred to herein, for example but without limitation, C₁₋₈ alkyl, the citation of a range should be considered a representation of each member of said range.

Other derivatives of the compounds of this invention have activity in both their acid and acid derivative forms, but in the acid sensitive form often offers advantages of solubility, tissue compatibility, or delayed release in the mammalian organism (Bundgard, H, 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides and anhydrides derived from acidic groups pendant on the compounds of this invention are particularly useful prodrugs. In some cases, it is desirable to prepare double ester type prodrugs such as (acyloxy)alkyl esters or ((alkoxycarbonyl)oxy)alkylesters. Particular such prodrugs are the C₁₋₈ alkyl, C₂₋₈ alkenyl, C₆₋₁₀ optionally substituted aryl, and (C₆₋₁₀ aryl)-(C₁₋₄ alkyl) esters of the compounds of the invention.

The present disclosure includes all isotopic forms of the compounds of the invention provided herein, whether in a form (i) wherein all atoms of a given atomic number have a mass number (or mixture of mass numbers) which predominates in nature (referred to herein as the “natural isotopic form”) or (ii) wherein one or more atoms are replaced by atoms having the same atomic number, but a mass number different from the mass number of atoms which predominates in nature (referred to herein as an “unnatural variant isotopic form”). It is understood that an atom may naturally exists as a mixture of mass numbers. The term “unnatural variant isotopic form” also includes embodiments in which the proportion of an atom of given atomic number having a mass number found less commonly in nature (referred to herein as an “uncommon isotope”) has been increased relative to that which is naturally occurring e.g. to the level of >20%, >50%, >75%, >90%, >95% or >99% by number of the atoms of that atomic number (the latter embodiment referred to as an “isotopically enriched variant form”). The term “unnatural variant isotopic form” also includes embodiments in which the proportion of an uncommon isotope has been reduced relative to that which is naturally occurring. Isotopic forms may include radioactive forms (i.e. they incorporate radioisotopes) and non-radioactive forms. Radioactive forms will typically be isotopically enriched variant forms.

An unnatural variant isotopic form of a compound may thus contain one or more artificial or uncommon isotopes such as deuterium (²H or D), carbon-11 (¹¹C), carbon-13 (¹³C), carbon-14 (¹⁴C), nitrogen-13 (¹³N), nitrogen-15 (¹⁵N), oxygen-15 (¹⁵O), oxygen-17 (¹⁷O), oxygen-18 (¹⁸O), phosphorus-32 (³²P), sulphur-35 (³⁵S), chlorine-36 (³⁶Cl), chlorine-37 (³⁷Cl), fluorine-18 (¹⁸F) iodine-123 (¹²³I), iodine-125 (¹²⁵I) in one or more atoms or may contain an increased proportion of said isotopes as compared with the proportion that predominates in nature in one or more atoms.

Unnatural variant isotopic forms comprising radioisotopes may, for example, be used for drug and/or substrate tissue distribution studies. The radioactive isotopes tritium, i.e. ³H, and carbon-14, i.e. ¹⁴C, are particularly useful for this purpose in view of their ease of incorporation and ready means of detection. Unnatural variant isotopic forms which incorporate deuterium i.e. ²H or D may afford certain therapeutic advantages resulting from greater metabolic stability, for example, increased in vivo half-life or reduced dosage requirements, and hence may be preferred in some circumstances. Further, unnatural variant isotopic forms may be prepared which incorporate positron emitting isotopes, such as ¹¹C, ¹⁸F, ¹⁵O and ¹³N, and would be useful in Positron Emission Topography (PET) studies for examining substrate receptor occupancy.

It is also to be understood that compounds that have the same molecular formula but differ in the nature or sequence of bonding of their atoms or the arrangement of their atoms in space are termed ‘isomers’. Isomers that differ in the arrangement of their atoms in space are termed ‘stereoisomers’.

Stereoisomers that are not mirror images of one another are termed cdiastereomers' and those that are non-superimposable mirror images of each other are termed ‘enantiomers’. When a compound has an asymmetric center, for example, it is bonded to four different groups, a pair of enantiomers is possible. An enantiomer can be characterized by the absolute configuration of its asymmetric center and is described by the R- and S-sequencing rules of Calm and Prelog, or by the manner in which the molecule rotates the plane of polarized light and designated as dextrorotatory or levorotatory (i.e. as (+) or (−)-isomers respectively). A chiral compound can exist as either individual enantiomer or as a mixture thereof. A mixture containing equal proportions of the enantiomers is called a ‘racemic mixture’.

‘Tautomers’ refer to compounds that are interchangeable forms of a particular compound structure, and that vary in the displacement of hydrogen atoms and electrons. Thus, two structures may be in equilibrium through the movement of π electrons and an atom (usually H). For example, enols and ketones are tautomers because they are rapidly interconverted by treatment with either acid or base. Another example of tautomerism is the aci- and nitro-forms of phenylnitromethane, that are likewise formed by treatment with acid or base.

Tautomeric forms may be relevant to the attainment of the optimal chemical reactivity and biological activity of a compound of interest.

The compounds of the invention may possess one or more asymmetric centers; such compounds can therefore be produced as individual (R)- or (S)-stereoisomers or as mixtures thereof.

Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art.

It will be appreciated that compounds of the invention may be metabolized to yield biologically active metabolites.

### THE INVENTION

Herein are provided inter alia, solid forms of (5S)-cyclopropyl-5-[3-[(3S)-4-(3,5-difluorophenyl)-3-methyl-piperazin-1-yl]-3-oxo-propyl]imidazolidine-2,4-dione of formula (I) and to pharmaceutical compositions comprising them and methods of their manufacture, as well as the use of said solid forms or compositions for the prophylaxis and/or treatment of inflammatory conditions, muscular disease, fibrotic diseases, viral infection, and/or diseases involving degradation of cartilage and/or disruption of cartilage homeostasis, especially osteoarthritis.

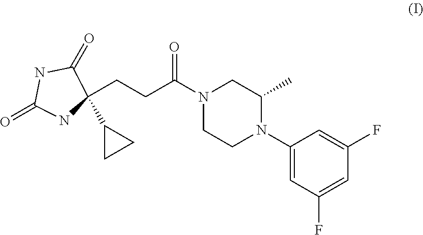 [SMILES: C[C@H]1CN(C(=O)CC[C@@]2(C3CC3)NC(=O)NC2=O)CCN1c1cc(F)cc(F)c1]

In one embodiment, the solid form of the invention is amorphous.

In one embodiment, the solid form of the invention is a crystalline form.

In one embodiment, the solid form of the invention is a solvate. In a particular embodiment, the solid form of the invention is a hydrate, or a dihydrate. In a more particular embodiment, the solid form of the invention is a dihydrate.

In one embodiment, the solid form of the invention is a unsolvated. In a particular embodiment, the solid form of the invention is anhydrous.

In one embodiment, the solid form of the invention is a dihydrate form of (5S)-cyclopropyl-5-[3-[(3S)-4-(3,5-difluorophenyl)-3-methyl-piperazin-1-yl]-3-oxo-propyl]imidazolidine-2,4-dione (Form I). In a most particular embodiment, the solid form of the invention is a crystalline dihydrate form of (5S)-cyclopropyl-5-[3-[(3S)-4-(3,5-difluorophenyl)-3-methyl-piperazin-1-yl]-3-oxo-propyl]imidazolidine-2,4-dione (Form I).

In another embodiment, the solid form of the invention is an anhydrous form of (5S)-cyclopropyl-5-[3-[(3S)-4-(3,5-difluorophenyl)-3-methyl-piperazin-1-yl]-3-oxo-propyl]imidaz olidine-2,4-dione (Form II). In a most particular embodiment, the solid form of the invention is a crystalline anhydrous form of (5S)-cyclopropyl-5-[3-[(3S)-4-(3,5-difluorophenyl)-3-methyl-piperazin-1-yl]-3-oxo-propyl]imidazolidine-2,4-dione (Form II).

### Amorphous Form

In one embodiment, a solid form of the invention is amorphous. In one embodiment, a solid form of the invention is characterized by an X-ray powder diffraction pattern substantially in accordance with FIG. 18.

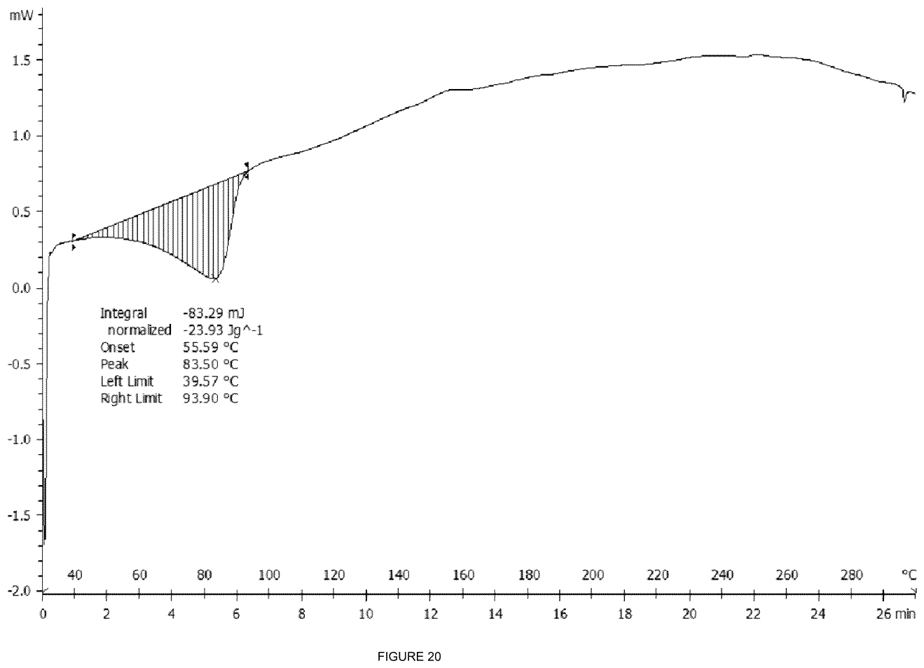

In one embodiment, the solid form of the invention is amorphous and further characterized by a DSC curve substantially in accordance with FIG. 20.

In one embodiment, the solid form of the invention is amorphous and characterized by an X-ray powder diffraction pattern substantially in accordance with FIG. 18 and a DSC curve substantially in accordance with FIG. 20.

### Polymorphic Form I

Provided herein inter alia is a dihydrate solid form of (5S)-cyclopropyl-5-[3-[(3S)-4-(3,5-difluorophenyl)-3-methyl-piperazin-1-yl]-3-oxo-propyl]imidazolidine-2,4-dione. In another embodiment, the solid form of the invention is crystalline (Form I). In yet another embodiment, the solid form of the invention is crystalline (Form I) and can be characterized by one or more of the parameters described in further detail below.

In one embodiment, a solid form of the invention is polymorphic form I and may be characterized by an X-ray powder diffraction pattern having one or more peaks at the following positions: 6.2, 12.5, 14.1, 14.6, 15.6, 15.7, 17.8, 18.0, 18.8, 19.1, 19.7, 20.8, 21.4, 22.4, 25.2, 26.4, 28.9, or 29.0±0.2° 2θ.

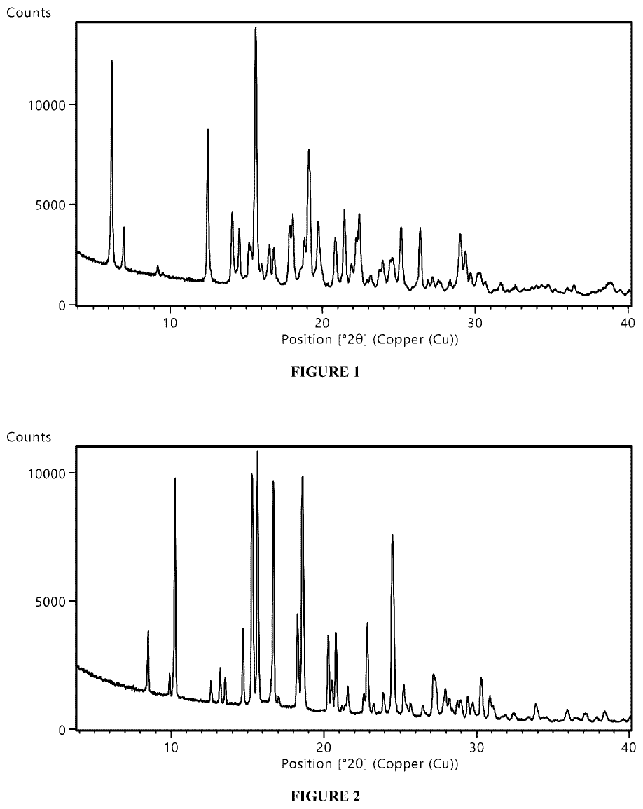

In a most particular embodiment, a solid form of the invention is polymorphic form I and may be characterized by an X-ray powder diffraction pattern having one or more peaks at the following positions: 6.2, 12.5, 14.1, 14.6, 15.6, 15.7, 17.8, 18.0, 18.8, 19.1, 19.7, 20.8, 21.4, 22.4, 25.2, 26.4, 28.9, or 29.0±0.2° 2θ; and an X-ray powder diffraction pattern substantially as depicted in FIG. 1.

In a particular embodiment, a solid form of the invention is polymorphic form I and may be characterized by an X-ray powder diffraction pattern having at least 1, 5, 10, 15 or more peaks at the following positions: 6.2, 12.5, 14.1, 14.6, 15.6, 15.7, 17.8, 18.0, 18.8, 19.1, 19.7, 20.8, 21.4, 22.4, 25.2, 26.4, 28.9, or 29.0±0.2° 2θ.

In a particular embodiment, a solid form of the invention is polymorphic form I and may be characterized by an X-ray powder diffraction pattern having peaks at the following positions: 6.2, 12.5, 15.7, 19.1, 25.2, and 26.4±0.2° 2θ.

In a particular embodiment, a solid form of the invention is polymorphic form I and may be characterized by an X-ray powder diffraction pattern having peaks at the following positions: 6.2, 12.5, 14.1, 15.7, 19.1, 21.4, 22.4, 25.2, and 26.4±0.2° 2θ.

In a particular embodiment, a solid form of the invention is polymorphic form I and may be characterized by an X-ray powder diffraction pattern having peaks at the following positions: 6.2, 12.5, 14.1, 14.6, 15.6, 15.7, 17.8, 18.0, 18.8, 19.1, 19.7, 20.8, 21.4, 22.4, 25.2, 26.4, 28.9, and 29.0±0.2° 2θ.

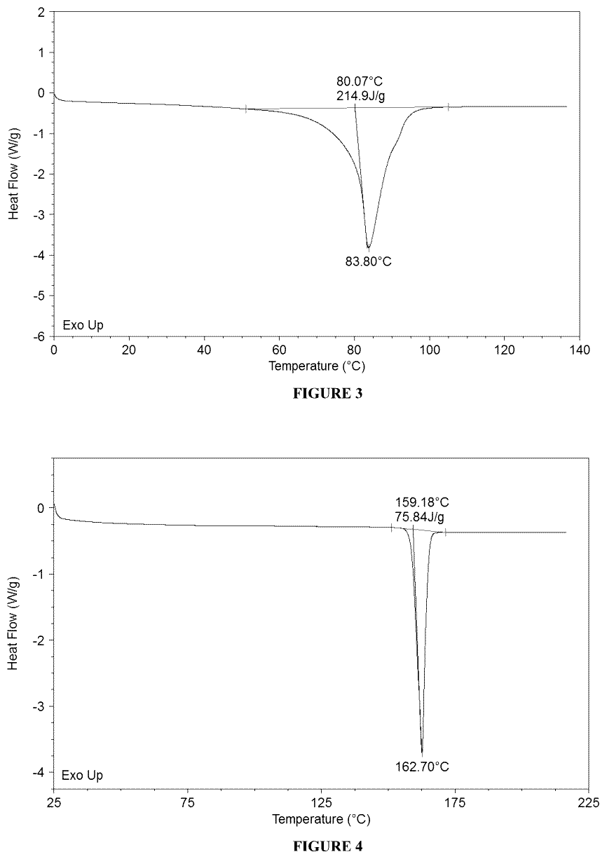

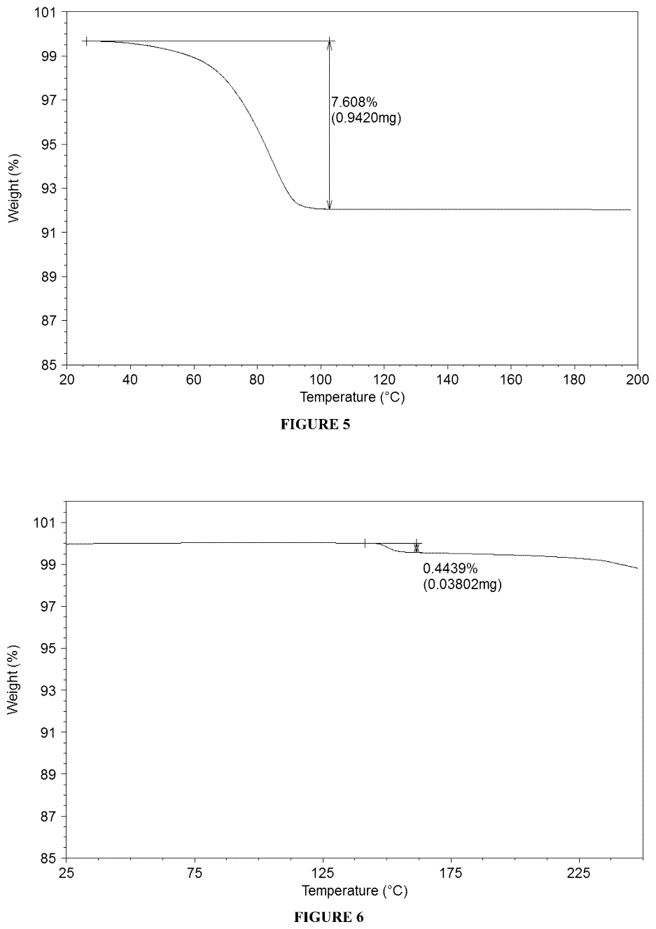

In one embodiment, a solid form of the invention is polymorphic form I and may be characterized by an X-ray powder diffraction pattern substantially as depicted in FIG. 1. In a particular embodiment, the solid form of the invention is polymorphic form I and is further characterized by the DSC profile on FIG. 3. In a particular embodiment, the solid form of the invention is polymorphic form I and is further characterized by the TGA profile on FIG. 5.

### Polymorphic Form II

Provided herein inter alia is an anhydrous solid form of (5S)-cyclopropyl-5-[3-[(3S)-4-(3,5-difluorophenyl)-3-methyl-piperazin-1-yl]-3-oxo-propyl]imidazolidine-2,4-dione. In another embodiment, the solid form of the invention is crystalline (Form II). In yet another embodiment, the solid form of the invention is crystalline (Form II) and can be characterized by one or more of the parameters described in further detail below.

In one embodiment, a solid form of the invention is polymorphic form II and may be characterized by an X-ray powder diffraction pattern having one or more peaks at the following positions: 8.5, 10.3, 12.6, 13.2, 13.6, 14.7, 15.3, 15.7, 16.7, 18.3, 18.6, 20.3, 20.8, 22.9, 24.5, 27.2, or 30.4±0.2° 2θ.

In one embodiment, a solid form of the invention is polymorphic form II and may be characterized by an X-ray powder diffraction pattern having at least 1, 5, 10, 15 or more peaks at the following positions: 8.5, 10.3, 12.6, 13.2, 13.6, 14.7, 15.3, 15.7, 16.7, 18.3, 18.6, 20.3, 20.8, 22.9, 24.5, 27.2, or 30.4±0.2° 2θ.

In one embodiment, a solid form of the invention is polymorphic form II and may be characterized by an X-ray powder diffraction pattern having peaks at the following positions: 10.3, 15.3, 15.7, 16.7, 18.6±0.2° 2θ.

In one embodiment, a solid form of the invention is polymorphic form II and may be characterized by an X-ray powder diffraction pattern having peaks at the following positions: 10.3, 15.3, 15.7, 16.7, 18.6, 24.5, 30.4 ±0.2° 2θ.

In one embodiment, a solid form of the invention is polymorphic form II and may be characterized by an X-ray powder diffraction pattern having peaks at the following positions: 8.5, 10.3, 12.6, 13.2, 13.6, 14.7, 15.3, 15.7, 16.7, 18.3, 18.6, 20.3, 20.8, 22.9, 24.5, 27.2, and 30.4, ±0.2° 2θ.

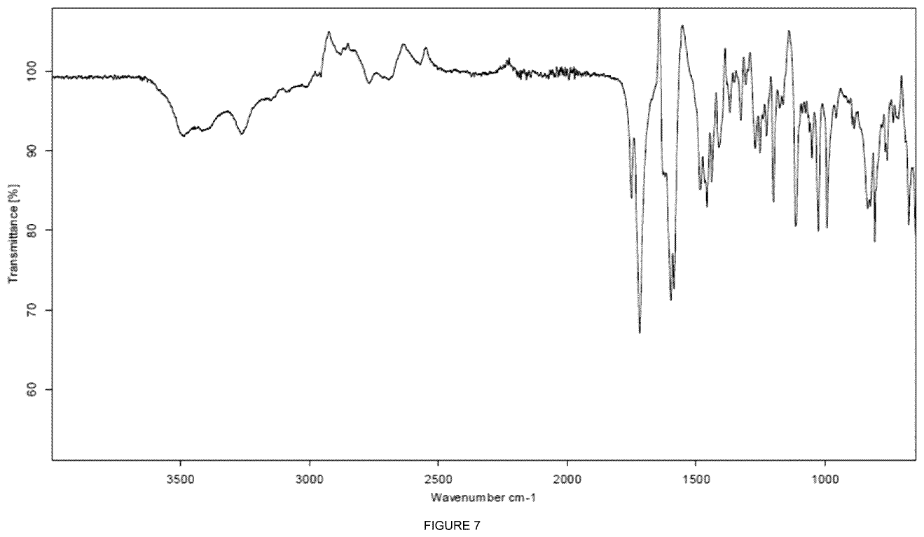

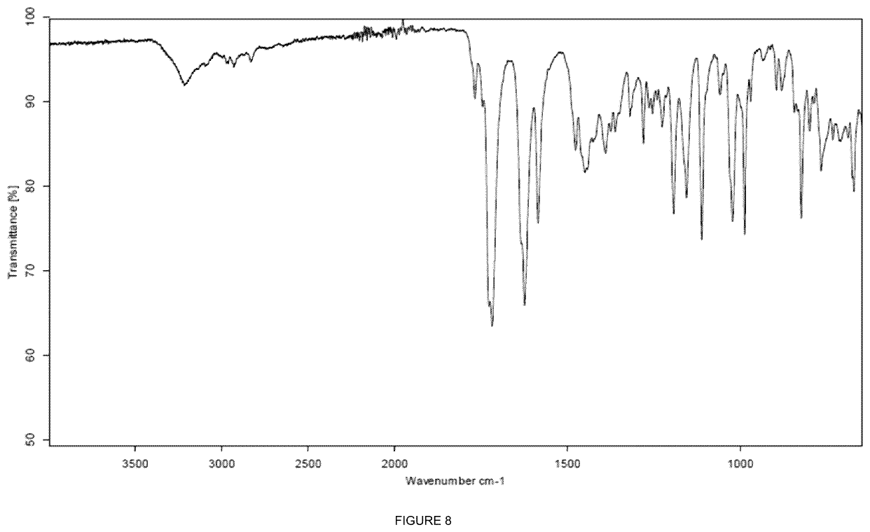

In one embodiment, a solid form of the invention is polymorphic form II and may be characterized by an X-ray powder diffraction pattern substantially as depicted in FIG. 2. In a particular embodiment, the solid form of the invention is polymorphic form II and is further characterized by the DSC profile on FIG. 4. In a more particular embodiment, the solid form of the invention is polymorphic form II and is further characterized by the TGA profile on FIG. 6.

### Polymorphic Form III

Provided herein inter alia is a solid form of (5S)-cyclopropyl-5-[3-[(3S)-4-(3,5-difluorophenyl)-3-methyl-piperazin-1-yl]-3-oxo-propyl]imidazolidine-2,4- dione. In another embodiment, the solid form of the invention is crystalline (Form III). In yet another embodiment, the solid form of the invention is crystalline (Form III) and can be characterized by one or more of the parameters described in further detail below.

In one embodiment, a solid form of the invention is polymorphic form III and may be characterized by an X-ray powder diffraction pattern having one or more peaks at the following positions: 9.0, 11.0, 11.4, 14.2, 15.0, 16.4, 16.6, 17.5, 18.1, 18.6, 18.8, 19.3, 20.0, 20.5, 21.7, 22.4, 23.4, 23.8, 26.2, 26.6, or 27.8±0.2° 2θ.

In a particular embodiment, a solid form of the invention is polymorphic form III and may be characterized by an X-ray powder diffraction pattern having at least 1, 5, 10, 15 or more peaks at the following positions: 9.0, 11.0, 11.4, 14.2, 15.0, 16.4, 16.6, 17.5, 18.1, 18.6, 18.8, 19.3, 20.0, 20.5, 21.7, 22.4, 23.4, 23.8, 26.2, 26.6, or 27.8±0.2° 2θ.

In a particular embodiment, a solid form of the invention is polymorphic form III and may be characterized by an X-ray powder diffraction pattern having peaks at the following positions: 11.0, 16.6, 17.5, 18.8, 20.5, and 22.4 ±0.2° 2θ.

In a particular embodiment, a solid form of the invention is polymorphic form III and may be characterized by an X-ray powder diffraction pattern having peaks at the following positions: 9.0, 11.0, 14.2, 16.4, 16.6, 17.5, 18.6, 18.8, 20.5, 22.4, 23.4, and 26.2,±0.2° 2θ.

In a particular embodiment, a solid form of the invention is polymorphic form III and may be characterized by an X-ray powder diffraction pattern having peaks at the following positions: 9.0, 11.0, 11.4, 14.2, 15.0, 16.4, 16.6, 17.5, 18.1, 18.6, 18.8, 19.3, 20.0, 20.5, 21.7, 22.4, 23.4, 23.8, 26.2, 26.6, and 27.8±0.2° 2θ.

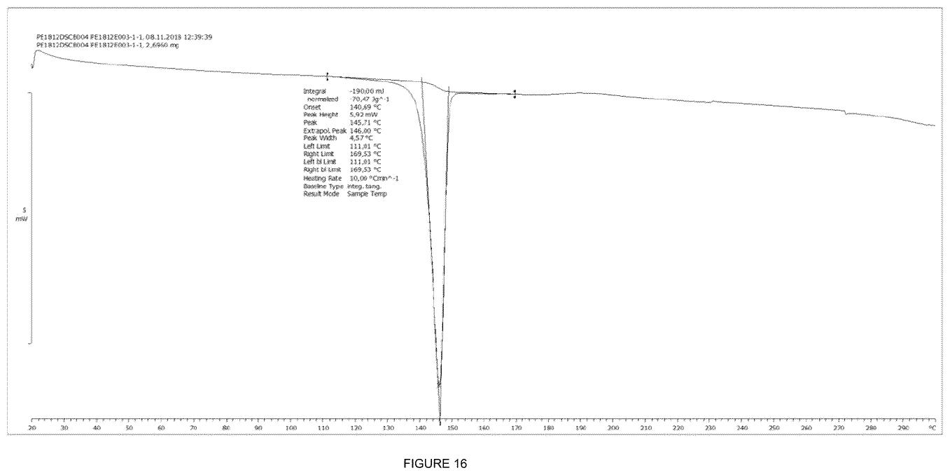

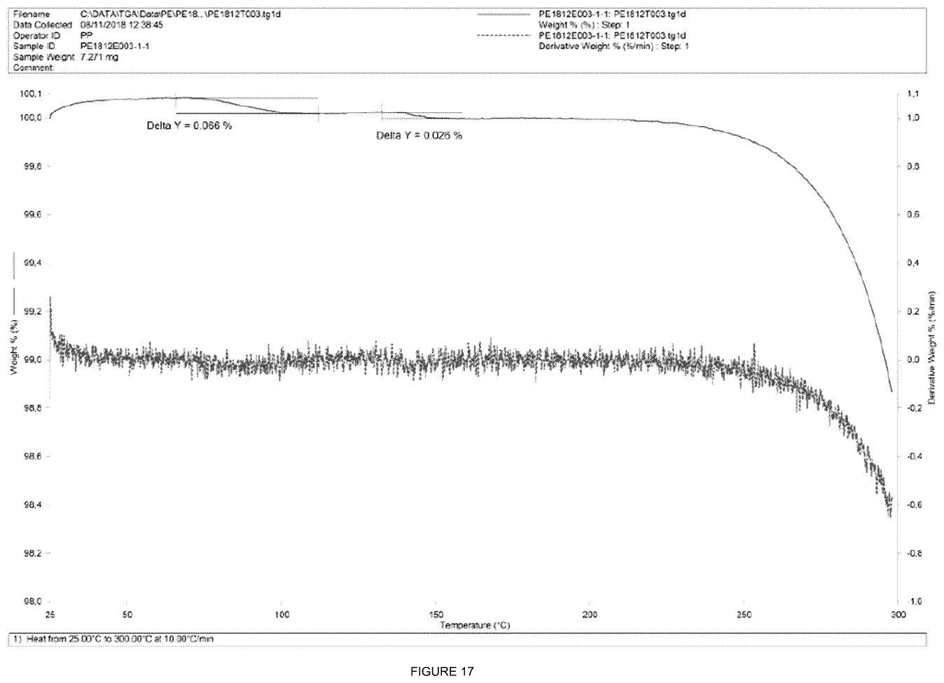

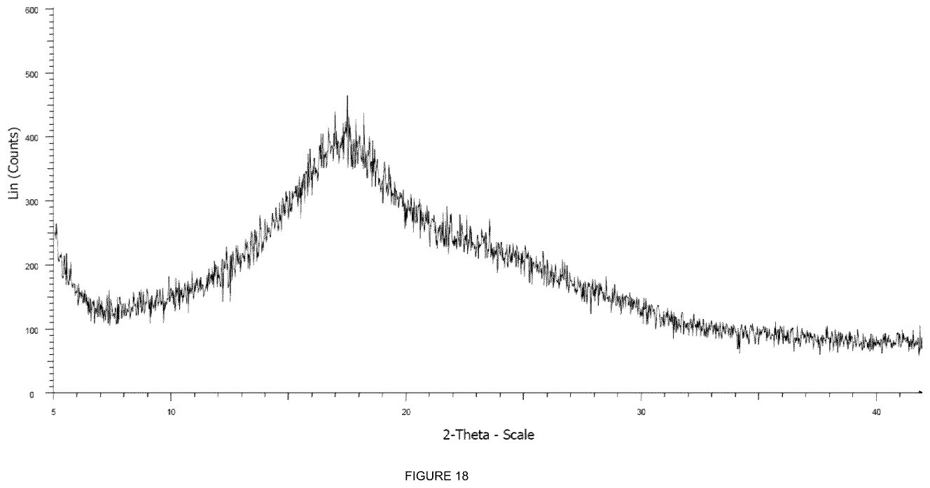

In one embodiment, a solid form of the invention is polymorphic form III and may be characterized by an X-ray powder diffraction pattern substantially as depicted in FIG. 15. In a particular embodiment, the solid form of the invention is polymorphic form III and is further characterized by the DSC profile on FIG. 16. In a more particular embodiment, the solid form of the invention is polymorphic form III and is further characterized by the TGA profile on FIG. 17.

### PHARMACEUTICAL COMPOSITIONS

In some embodiments, solid forms disclosed herein can be included in a solid dosage form, such as a tablet. For example, in some embodiments, a solid form disclosed herein can be provided in a tablet in amount ranging from 50 mg to 1000 mg. In some embodiments, a tablet comprising 50 mg to 1000 mg polymorphic form I is provided. In some embodiments, a tablet comprising 50 mg to 1000 mg polymorphic form II is provided. In some embodiments, a tablet comprising 50 mg to 1000 mg polymorphic form III is provided. In some embodiments, a tablet comprising 50 mg to 1000 mg amorphous form is provided.

In one embodiment, herein is provided inter alia, a pharmaceutical composition comprising a solid form of the invention and an inert solid diluent. In a particular embodiment, herein is provided inter alia, a pharmaceutical composition comprising 29-31% wt of polymorphic form I and 42-43% wt of an inert solid diluent. In a particular embodiment, the solid form of the invention is polymorphic form I, or polymorphic form II. In another particular embodiment, the inert solid diluent is lactose monohydrate. form of the invention, a first inert solid diluent and a second inert solid diluent. In a particular embodiment, herein is provided inter alia, a pharmaceutical composition comprising 29-31% wt of polymorphic form I, 42-43% wt of a first inert solid diluent, and 19.5-20.5% wt of a second inert diluent. In a particular embodiment, the solid form of the invention is polymorphic form I, or polymorphic form II. In a more particular embodiment, the first inert solid diluent is lactose monohydrate. In another more particular embodiment, the first inert solid diluent is corn starch.

In one embodiment, herein is provided inter alia, a pharmaceutical composition comprising a solid form of the invention, a first inert solid diluent and a second inert solid diluent. In a particular embodiment, herein is provided inter alia, a pharmaceutical composition comprising 29-31% wt of polymorphic form I, 42-43% wt of a first inert solid diluent, 19.5-20.5% wt of a second inert diluent and 6.5-7.5% wt of a disintegrant. In a particular embodiment, the solid form of the invention is polymorphic form I, or polymorphic form II. In a more particular embodiment, the first inert solid diluent is lactose monohydrate. In another more particular embodiment, the first inert solid diluent is corn starch. In yet another more particular embodiment, the disintegrant is povidone.

In one embodiment, herein is provided inter alia, a pharmaceutical composition comprising a solid form of the invention, a first inert solid diluent and a second inert solid diluent. In a particular embodiment, herein is provided inter alia, a pharmaceutical composition comprising 29-31% wt of polymorphic form I, 42-43% wt of a first inert solid diluent, 19.5-20.5% wt of a second inert diluent, 6.5-7.5% wt of a disintegrant, and 0.15-0.25% wt of a glidant. In a particular embodiment, the solid form of the invention is polymorphic form I, or polymorphic form II. In a more particular embodiment, the first inert solid diluent is lactose monohydrate. In another more particular embodiment, the first inert solid diluent is corn starch. In yet another more particular embodiment, the disintegrant is povidone. In yet another more particular embodiment, the glidant is colloidal silicon dioxide.

In a particular embodiment, herein is provided inter alia a pharmaceutical composition comprising in weight:
1) 29-31% wt of solid form of the invention,
2) 42-43% wt of lactose monohydrate,
3) 19.5-20.5% wt Corn starch
4) 6.5-7.5% wt povidone
5) 0.15-0.25% wt colloidal silicon dioxide
6) 0.4-0.6% wt magnesium stearate

In a particular embodiment, herein is provided inter alia a pharmaceutical composition comprising in weight:
1) About 30% wt of solid form of the invention,
2) About 42.3% wt of lactose monohydrate,
3) About 20% wt Corn starch
4) About 7% wt povidone
5) About 0.2% wt colloidal silicon dioxide
6) About 0.5% wt magnesium stearate

In a particular embodiment, herein is provided inter alia a pharmaceutical composition comprising in weight:
1) 29-31% wt of polymorphic form II,
2) 42-43% wt of lactose monohydrate,
3) 19.5-20.5% wt Corn starch
4) 6.5 -7.5% wt povidone
5) 0.15-0.25% wt colloidal silicon dioxide
6) 0.4-0.6% wt magnesium stearate

In a particular embodiment, herein is provided inter alia a pharmaceutical composition comprising in weight:
1) About 30% wt of polymorphic form II,
2) About 42.3% wt of lactose monohydrate,
3) About 20% wt Corn starch
4) About 7% wt povidone
5) About 0.2% wt colloidal silicon dioxide
6) About 0.5% wt magnesium stearate

In one embodiment, herein is provided inter alia, a pharmaceutical composition comprising a solid form of the invention and an inert solid diluent. In a particular embodiment, herein is provided inter alia, a pharmaceutical composition comprising 49-51% wt of polymorphic form I and 22-23% wt of an inert solid diluent. In a particular embodiment, the solid form of the invention is polymorphic form I, or polymorphic form II. In another particular embodiment, the inert solid diluent is lactose monohydrate.

In one embodiment, herein is provided inter alia, a pharmaceutical composition comprising a solid form of the invention, a first inert solid diluent and a second inert solid diluent. In a particular embodiment, herein is provided inter alia, a pharmaceutical composition comprising 49-51% wt of polymorphic form I, 22-23% wt of a first inert solid diluent, and 19.5-20.5% wt of a second inert diluent. In a particular embodiment, the solid form of the invention is polymorphic form I, or polymorphic form II. In a more particular embodiment, the first inert solid diluent is lactose monohydrate. In another more particular embodiment, the first inert solid diluent is corn starch.

In one embodiment, herein is provided inter alia, a pharmaceutical composition comprising a solid form of the invention, a first inert solid diluent and a second inert solid diluent. In a particular embodiment, herein is provided inter alia, a pharmaceutical composition comprising 49-51% wt of polymorphic form I, 22-23% wt of a first inert solid diluent, 19.5-20.5% wt of a second inert diluent and 6.5-7.5% wt of a disintegrant. In a particular embodiment, the solid form of the invention is polymorphic form I, or polymorphic form II. In a more particular embodiment, the first inert solid diluent is lactose monohydrate. In another more particular embodiment, the first inert solid diluent is corn starch. In yet another more particular embodiment, the disintegrant is povidone.

In one embodiment, herein is provided inter alia, a pharmaceutical composition comprising a solid form of the invention, a first inert solid diluent and a second inert solid diluent. In a particular embodiment, herein is provided inter alia, a pharmaceutical composition comprising 49-51% wt of polymorphic form I, 22-23% wt of a first inert solid diluent, 19.5-20.5% wt of a second inert diluent, 6.5-7.5% wt of a disintegrant, and 0.15-0.25% wt of a glidant. In a particular embodiment, the solid form of the invention is polymorphic form I, or polymorphic form II. In a more particular embodiment, the first inert solid diluent is lactose monohydrate. In another more particular embodiment, the first inert solid diluent is corn starch. In yet another more particular embodiment, the disintegrant is povidone. In yet another more particular embodiment, the glidant is colloidal silicon dioxide.

In a particular embodiment, herein is provided inter alia a pharmaceutical composition comprising in weight:
1) 49-51% wt of solid form of the invention,
2) 22-23% wt of lactose monohydrate,(diluent)
3) 19.5-20.5% wt Corn starch (diluent)
4) 6.5-7.5% wt povidone (disintegrant)
5) 0.15-0.25% wt colloidal silicon dioxide (glidant)
6) 0.4-0.6% wt magnesium stearate (lubricant)

In a particular embodiment, herein is provided inter alia a pharmaceutical composition comprising in weight:
1) About 50% wt of solid form of the invention,
2) About 22.3% wt of lactose monohydrate,
3) About 20% wt Corn starch
4) About 7% wt povidone
5) About 0.2% wt colloidal silicon dioxide
6) About 0.5% wt magnesium stearate

In a particular embodiment, herein is provided inter alia a pharmaceutical composition comprising in weight:
1) 49-51% wt of polymorphic form II,
2) 22-23% wt of lactose monohydrate,(diluent)
3) 19.5-20.5% wt Corn starch (diluent)
4) 6.5-7.5% wt povidone (disintegrant)
5) 0.15-0.25% wt colloidal silicon dioxide (glidant)
6) 0.4-0.6% wt magnesium stearate (lubricant)

In a particular embodiment, herein is provided inter alia a pharmaceutical composition comprising in weight:
1) About 50% wt of polymorphic form II,
2) About 22.3% wt of lactose monohydrate,
3) About 20% wt Corn starch
4) About 7% wt povidone
5) About 0.2% wt colloidal silicon dioxide
6) About 0.5% wt magnesium stearate

When employed as a pharmaceutical, a compound of the invention is typically administered in the form of a pharmaceutical composition. Such compositions can be prepared in a manner well known in the pharmaceutical art and comprise at least one active compound of the invention according to Formula I. Generally, a compound of the invention is administered in a pharmaceutically effective amount. The amount of compound of the invention actually administered will typically be determined by a physician, in the light of the relevant circumstances, including the condition to be treated, the chosen route of administration, the actual compound of the invention administered, the age, weight, and response of the individual patient, the severity of the patient's symptoms, and the like.

The pharmaceutical compositions of this invention can be administered by a variety of routes including oral, rectal, transdermal, subcutaneous, intra-articular, intravenous, intramuscular, and intranasal. Depending on the intended route of delivery, a compound of the invention is preferably formulated as either injectable or oral compositions or as salves, as lotions or as patches all for transdermal administration.

The compositions for oral administration can take the form of bulk liquid solutions or suspensions, or bulk powders. More commonly, however, the compositions are presented in unit dosage forms to facilitate accurate dosing. The term ‘unit dosage forms’ refers to physically discrete units suitable as unitary dosages for human subjects and other mammals, each unit containing a predetermined quantity of active material calculated to produce the desired therapeutic effect, in association with a suitable pharmaceutical excipient, vehicle or carrier. Typical unit dosage forms include prefilled, premeasured ampules or syringes of the liquid compositions or pills, tablets, capsules or the like in the case of solid compositions. In such compositions, the compound of the invention according to Formula I is usually a minor component (from about 0.1 to about 50% by weight or preferably from about 1 to about 40% by weight) with the remainder being various vehicles or carriers and processing aids helpful for forming the desired dosing form.

Liquid forms suitable for oral administration may include a suitable aqueous or non-aqueous vehicle with buffers, suspending and dispensing agents, colorants, flavors and the like. Solid forms may include, for example, any of the following ingredients, or compound of the inventions of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint or orange flavoring.

Injectable compositions are typically based upon injectable sterile saline or phosphate-buffered saline or other injectable carriers known in the art. As before, the active compound of the invention according to Formula I in such compositions is typically a minor component, often being from about 0.05 to 10% by weight with the remainder being the injectable carrier and the like.

Transdermal compositions are typically formulated as a topical ointment or cream containing the active ingredient(s), generally in an amount ranging from about 0.01 to about 20% by weight, preferably from about 0.1 to about 20% by weight, preferably from about 0.1 to about 10% by weight, and more preferably from about 0.5 to about 15% by weight. When formulated as an ointment, the active ingredients will typically be combined with either a paraffinic or a water-miscible ointment base. Alternatively, the active ingredients may be formulated in a cream with, for example an oil-in-water cream base. Such transdermal formulations are well-known in the art and generally include additional ingredients to enhance the dermal penetration of stability of the active ingredients or the formulation. All such known transdermal formulations and ingredients are included within the scope of this invention.

A compound of the invention can also be administered by a transdermal device. Accordingly, transdermal administration can be accomplished using a patch either of the reservoir or porous membrane type, or of a solid matrix variety.

The above-described components for orally administrable, injectable or topically administrable compositions are merely representative. Other materials as well as processing techniques and the like are set forth in Part 8 of Remington's Pharmaceutical Sciences, 17^{th} edition, 1985, Mack Publishing Company, Easton, Pennsylvania, which is incorporated herein by reference.

A compound of the invention can also be administered in sustained release forms or from sustained release drug delivery systems. A description of representative sustained release materials can be found in Remington's Pharmaceutical Sciences.

The following formulation examples illustrate representative pharmaceutical compositions that may be prepared in accordance with this invention. The present invention, however, is not limited to the following pharmaceutical compositions.

### Formulation 1—Tablets

A compound of the invention according to Formula I may be admixed as a dry powder with a dry gelatin binder in an approximate 1:2 weight ratio. A minor amount of magnesium stearate may be added as a lubricant. The mixture may be formed into 240-270 mg tablets (75 mg of active compound of the invention according to Formula I per tablet) in a tablet press.

### Formulation 2—Capsules

A compound of the invention according to Formula I may be admixed as a dry powder with a starch diluent in an approximate 1:1 weight ratio. The mixture may be filled into 250 mg capsules (125 mg of active compound of the invention according to Formula I per capsule).

### Formulation 3—Liquid

A compound of the invention according to Formula I (125 mg), may be admixed with sucrose (1.75 g) and xanthan gum (4 mg) and the resultant mixture may be blended, passed through a No. 10 mesh U.S. sieve, and then mixed with a previously made solution of microcrystalline cellulose and sodium carboxymethyl cellulose (11:89, 50 mg) in water. Sodium benzoate (10 mg), flavor, and color may be diluted with water and added with stirring. Sufficient water may then be added with stirring. Further sufficient water may be then added to produce a total volume of 5 mL.

### Formulation 4—Tablets

A compound of the invention according to Formula I may be admixed as a dry powder with a dry gelatin binder in an approximate 1:2 weight ratio. A minor amount of magnesium stearate may be added as a lubricant. The mixture may be formed into 450-900 mg tablets (150-300 mg of active compound of the invention according to Formula I) in a tablet press.

### Illustrative Example—Tablets

Cpd 1 (50 g), lactose monohydrate (22.5 g), microcrystalline cellulose (23 g), colloidal silica anhydrous (0.5g) and croscarmellose sodium (3 g) are weighed individually, transfered to a suitable recipient and mixed.

The resulting powder is dry granulated using roller compaction. The granules are sieved on a 500 μm sieve and magnesium stearate (0.5 g) is added and blended.

The final blend is then compressed into tablets using a tablet press applying ±20 N force.

### Formulation 5—Injection

A compound of the invention according to Formula I may be dissolved or suspended in a buffered sterile saline injectable aqueous medium to a concentration of approximately 5 mg/mL.

### Formulation 6—Topical

Stearyl alcohol (250 g) and a white petrolatum (250 g) may be melted at about 75° C. and then a mixture of A compound of the invention according to Formula I (50 g) methylparaben (0.25 g), propylparaben (0.15 g), sodium lauryl sulfate (10 g), and propylene glycol (120 g) dissolved in water (about 370 g) may be added and the resulting mixture may be stirred until it congeals.

### METHOD OF TREATMENT

In one embodiment, the present invention provides compounds of the invention or pharmaceutical compositions comprising a compound of the invention, for use in the prophylaxis and/or treatment of inflammatory diseases. In particular, the term refers to rheumatoid arthritis, osteoarthritis, juvenile idiopathic arthritis, psoriasis, psoriatic arthritis, ankylosing spondylitis, allergic airway disease (e.g. asthma, rhinitis), chronic obstructive pulmonary disease (COPD), inflammatory bowel diseases (e.g. Crohn's disease, ulcerative colitis), endotoxin-driven disease states (e.g. complications after bypass surgery or chronic endotoxin states contributing to e.g. chronic cardiac failure), and related diseases involving cartilage, such as that of the joints. More particularly, the term refers to rheumatoid arthritis, osteoarthritis, asthma, chronic obstructive pulmonary disease (COPD) and inflammatory bowel diseases (e.g. Crohn's disease, ulcerative colitis).

In another embodiment, the present invention provides the use of compounds of the invention or pharmaceutical compositions comprising a compound of the invention in the manufacture of a medicament for the prophylaxis and/or treatment of inflammatory diseases. In particular, the term refers to rheumatoid arthritis, osteoarthritis, juvenile idiopathic arthritis, psoriasis, psoriatic arthritis, ankylosing spondylitis, allergic airway disease (e.g. asthma, rhinitis), chronic obstructive pulmonary disease (COPD), inflammatory bowel diseases (e.g. Crohn's disease, ulcerative colitis), endotoxin-driven disease states (e.g. complications after bypass surgery or chronic endotoxin states contributing to e.g. chronic cardiac failure), and related diseases involving cartilage, such as that of the joints. More particularly, the term refers to rheumatoid arthritis, osteoarthritis, asthma, chronic obstructive pulmonary disease (COPD) and inflammatory bowel diseases (e.g. Crohn's disease, ulcerative colitis).

In additional method of treatment aspects, this invention provides methods of prophylaxis and/or treatment of a mammal afflicted with inflammatory diseases, which methods comprise the administration of an effective amount of a compound of the invention or one or more of the pharmaceutical compositions herein described for the treatment or prophylaxis of said condition. In particular, the term refers to rheumatoid arthritis, osteoarthritis, juvenile idiopathic arthritis, psoriasis, psoriatic arthritis, ankylosing spondylitis, allergic airway disease (e.g. asthma, rhinitis), chronic obstructive pulmonary disease (COPD), inflammatory bowel diseases (e.g. Crohn's disease, ulcerative colitis), endotoxin-driven disease states (e.g. complications after bypass surgery or chronic endotoxin states contributing to e.g. chronic cardiac failure), and related diseases involving cartilage, such as that of the joints. More particularly, the term refers to rheumatoid arthritis, osteoarthritis, asthma, chronic obstructive pulmonary disease (COPD) and inflammatory bowel diseases (e.g. Crohn's disease, ulcerative colitis).

In one embodiment, the present invention provides pharmaceutical compositions comprising a compound of the invention, and another therapeutic agent. In a particular embodiment, the other therapeutic agent is an inflammatory diseases treatment agent. In particular, the term refers to rheumatoid arthritis, osteoarthritis, juvenile idiopathic arthritis, psoriasis, psoriatic arthritis, ankylosing spondylitis, allergic airway disease (e.g. asthma, rhinitis), chronic obstructive pulmonary disease (COPD), inflammatory bowel diseases (e.g. Crohn's disease, ulcerative colitis), endotoxin-driven disease states (e.g. complications after bypass surgery or chronic endotoxin states contributing to e.g. chronic cardiac failure), and related diseases involving cartilage, such as that of the joints. More particularly, the term refers to rheumatoid arthritis, osteoarthritis, asthma, chronic obstructive pulmonary disease (COPD) and inflammatory bowel diseases (e.g. Crohn's disease, ulcerative colitis).

In one embodiment, the present invention provides compounds of the invention or pharmaceutical compositions comprising a compound of the invention, for use in the prophylaxis and/or treatment of muscular disease. In particular, the term refers to muscular dystrophy. More particularly, the term refers to Duchenne type muscular dystrophy, Becker muscular dystrophy, myotonic dystrophy, facioscapulohumeral dystrophy, congenital dystrophy, and/or limb-girdle dystrophy. Most particularly, the term refers to Duchenne type muscular dystrophy.

In another embodiment, the present invention provides the use of compounds of the invention or pharmaceutical compositions comprising a compound of the invention in the manufacture of a medicament for the prophylaxis and/or treatment of muscular disease. In particular, the term refers to muscular dystrophy. More particularly, the term refers to Duchenne type muscular dystrophy, Becker muscular dystrophy, myotonic dystrophy, facioscapulohumeral dystrophy, congenital dystrophy, and/or limb-girdle dystrophy. Most particularly, the term refers to Duchenne type muscular dystrophy.

In additional method of treatment aspects, this invention provides methods of prophylaxis and/or treatment of a mammal afflicted with muscular disease, which methods comprise the administration of an effective amount of a compound of the invention or one or more of the pharmaceutical compositions herein described for the treatment or prophylaxis of said condition. In particular, the term refers to muscular dystrophy. More particularly, the term refers to Duchenne type muscular dystrophy, Becker muscular dystrophy, myotonic dystrophy, facioscapulohumeral dystrophy, congenital dystrophy, and/or limb-girdle dystrophy. Most particularly, the term refers to Duchenne type muscular dystrophy.

In one embodiment, the present invention provides pharmaceutical compositions comprising a compound of the invention, and another therapeutic agent. In a particular embodiment, the other therapeutic agent is a muscular disease treatment agent. In particular, the term refers to muscular dystrophy. More particularly, the term refers to Duchenne type muscular dystrophy, Becker muscular dystrophy, myotonic dystrophy, facioscapulohumeral dystrophy, congenital dystrophy, and/ or limb-girdle dystrophy. Most particularly, the term refers to Duchenne type muscular dystrophy.

In one embodiment, the present invention provides compounds of the invention or pharmaceutical compositions comprising a compound of the invention, for use in the prophylaxis and/or treatment of fibrotic diseases. In particular, the term refers to fibrosis of individual organs or tissues such as the heart, kidney, liver, joints, lung, pleural tissue, peritoneal tissue, skin, cornea, retina, musculoskeletal and digestive tract. In particular, the term fibrotic diseases refers to pulmonary fibrosis (such as idiopathic pulmonary fibrosis (IPF), progressive massive fibrosis (PMF), and/or cystic fibrosis (CF)), other diffuse parenchymal lung diseases of different etiologies including iatrogenic drug-induced fibrosis, occupational and/or environmental induced fibrosis, granulomatous diseases (sarcoidosis, hypersensitivity pneumonia), collagen vascular disease, alveolar proteinosis, langerhans cell granulomatosis, lymphangioleiomyomatosis, inherited diseases (Hermansky-Pudlak Syndrome, tuberous sclerosis, neurofibromatosis, metabolic storage disorders, familial interstitial lung disease); radiation induced fibrosis; chronic obstructive pulmonary disease (COPD); scleroderma; bleomycin induced pulmonary fibrosis; chronic asthma; silicosis; asbestos induced pulmonary fibrosis; acute respiratory distress syndrome (ARDS); kidney fibrosis; polycystic disease (PKD), autosomal dominant polycystic kidney disease (ADPKD), tubulointerstitium fibrosis; glomerular nephritis; focal segmental glomerular sclerosis; IgA nephropathy, membranous nephropathy; hypertension; Alport; gut fibrosis; liver fibrosis; cirrhosis; alcohol induced liver fibrosis; toxic/drug induced liver fibrosis; hemochromatosis; nonalcoholic steatohepatitis (NASH); biliary duct injury; primary biliary cirrhosis; infection induced liver fibrosis; viral induced liver fibrosis; and autoimmune hepatitis; corneal scarring; hypertrophic scarring; Dupuytren disease, keloids, cutaneous fibrosis; cutaneous scleroderma; systemic sclerosis, spinal cord injury/fibrosis; myelofibrosis; vascular restenosis; atherosclerosis; arteriosclerosis; Wegener's granulomatosis; Peyronie's disease, or chronic lymphocytic. More particularly, the term refers to idiopathic pulmonary fibrosis (IPF), IgA Nephropathy, Membranous Nephropathy, focal segmental glomerulo sclerosis, autosomal dominant polycystic kidney disease (ADPKD), and/or nonalcoholic steatohepatitis (NASH). Most particularly, the term refers to idiopathic pulmonary fibrosis (IPF), IgA Nephropathy, autosomal dominant polycystic kidney disease (ADPKD), and/or nonalcoholic steatohepatitis (NASH).

In another embodiment, the present invention provides the use of compounds of the invention or pharmaceutical compositions comprising a compound of the invention in the manufacture of a medicament for the prophylaxis and/or treatment of fibrotic diseases. In particular, the term refers to fibrosis of individual organs or tissues such as the heart, kidney, liver, joints, lung, pleural tissue, peritoneal tissue, skin, cornea, retina, musculoskeletal and digestive tract. In particular, the term fibrotic diseases refers to pulmonary fibrosis (such as idiopathic pulmonary fibrosis (IPF), progressive massive fibrosis (PMF), and/or cystic fibrosis (CF)), other diffuse parenchymal lung diseases of different etiologies including iatrogenic drug-induced fibrosis, occupational and/or environmental induced fibrosis, granulomatous diseases (sarcoidosis, hypersensitivity pneumonia), collagen vascular disease, alveolar proteinosis, langerhans cell granulomatosis, lymphangioleiomyomatosis, inherited diseases (Hermansky-Pudlak Syndrome, tuberous sclerosis, neurofibromatosis, metabolic storage disorders, familial interstitial lung disease); radiation induced fibrosis; chronic obstructive pulmonary disease (COPD); scleroderma; bleomycin induced pulmonary fibrosis; chronic asthma; silicosis; asbestos induced pulmonary fibrosis; acute respiratory distress syndrome (ARDS); kidney fibrosis; polycystic disease (PKD), autosomal dominant polycystic kidney disease (ADPKD), tubulointerstitium fibrosis; glomerular nephritis; focal segmental glomerular sclerosis; IgA nephropathy, membranous nephropathy; hypertension; Alport; gut fibrosis; liver fibrosis; cirrhosis; alcohol induced liver fibrosis; toxic/drug induced liver fibrosis; hemochromatosis; nonalcoholic steatohepatitis (NASH); biliary duct injury; primary biliary cirrhosis; infection induced liver fibrosis; viral induced liver fibrosis; and autoimmune hepatitis; corneal scarring; hypertrophic scarring; Dupuytren disease, keloids, cutaneous fibrosis; cutaneous scleroderma; systemic sclerosis, spinal cord injury/fibrosis; myelofibrosis; vascular restenosis; atherosclerosis; arteriosclerosis; Wegener's granulomatosis; Peyronie's disease, or chronic lymphocytic. More particularly, the term refers to idiopathic pulmonary fibrosis (IPF), IgA Nephropathy, Membranous Nephropathy, focal segmental glomerulo sclerosis , autosomal dominant polycystic kidney disease (ADPKD), and/or nonalcoholic steatohepatitis (NASH). Most particularly, the term refers to idiopathic pulmonary fibrosis (IPF), IgA Nephropathy, autosomal dominant polycystic kidney disease (ADPKD), and/or nonalcoholic steatohepatitis (NASH).

In additional method of treatment aspects, this invention provides methods of prophylaxis and/or treatment of a mammal afflicted with fibrotic diseases, which methods comprise the administration of an effective amount of a compound of the invention or one or more of the pharmaceutical compositions herein described for the treatment or prophylaxis of said condition. In particular, the term refers to fibrosis of individual organs or tissues such as the heart, kidney, liver, joints, lung, pleural tissue, peritoneal tissue, skin, cornea, retina, musculoskeletal and digestive tract. In particular, the term fibrotic diseases refers to pulmonary fibrosis (such as idiopathic pulmonary fibrosis (IPF), progressive massive fibrosis (PMF), and/or cystic fibrosis (CF)), other diffuse parenchymal lung diseases of different etiologies including iatrogenic drug-induced fibrosis, occupational and/or environmental induced fibrosis, granulomatous diseases (sarcoidosis, hypersensitivity pneumonia), collagen vascular disease, alveolar proteinosis, langerhans cell granulomatosis, lymphangioleiomyomatosis, inherited diseases (Hermansky-Pudlak Syndrome, tuberous sclerosis, neurofibromatosis, metabolic storage disorders, familial interstitial lung disease); radiation induced fibrosis; chronic obstructive pulmonary disease (COPD); scleroderma; bleomycin induced pulmonary fibrosis; chronic asthma; silicosis; asbestos induced pulmonary fibrosis; acute respiratory distress syndrome (ARDS); kidney fibrosis; polycystic disease (PKD), autosomal dominant polycystic kidney disease (ADPKD), tubulointerstitium fibrosis; glomerular nephritis; focal segmental glomerular sclerosis; IgA nephropathy, membranous nephropathy; hypertension; Alport; gut fibrosis; liver fibrosis; cirrhosis; alcohol induced liver fibrosis; toxic/drug induced liver fibrosis; hemochromatosis; nonalcoholic steatohepatitis (NASH); biliary duct injury; primary biliary cirrhosis; infection induced liver fibrosis; viral induced liver fibrosis; and autoimmune hepatitis; corneal scarring; hypertrophic scarring; Dupuytren disease, keloids, cutaneous fibrosis; cutaneous scleroderma; systemic sclerosis, spinal cord injury/fibrosis; myelofibrosis; vascular restenosis; atherosclerosis; arteriosclerosis; Wegener's granulomatosis; Peyronie's disease, or chronic lymphocytic. More particularly, the term refers to idiopathic pulmonary fibrosis (IPF), IgA Nephropathy, Membranous Nephropathy, focal segmental glomerulo sclerosis, autosomal dominant polycystic kidney disease (ADPKD), and/or nonalcoholic steatohepatitis (NASH). Most particularly, the term refers to idiopathic pulmonary fibrosis (IPF), IgA Nephropathy, autosomal dominant polycystic kidney disease (ADPKD), and/or nonalcoholic steatohepatitis (NASH).

In one embodiment, the present invention provides pharmaceutical compositions comprising a compound of the invention, and another therapeutic agent. In a particular embodiment, the other therapeutic agent is a fibrotic diseases treatment agent. In particular, the term refers to fibrosis of individual organs or tissues such as the heart, kidney, liver, joints, lung, pleural tissue, peritoneal tissue, skin, cornea, retina, musculoskeletal and digestive tract. In particular, the term fibrotic diseases refers to pulmonary fibrosis (such as idiopathic pulmonary fibrosis (IPF), progressive massive fibrosis (PMF), and/or cystic fibrosis (CF)), other diffuse parenchymal lung diseases of different etiologies including iatrogenic drug-induced fibrosis, occupational and/or environmental induced fibrosis, granulomatous diseases (sarcoidosis, hypersensitivity pneumonia), collagen vascular disease, alveolar proteinosis, langerhans cell granulomatosis, lymphangioleiomyomatosis, inherited diseases (Hermansky-Pudlak Syndrome, tuberous sclerosis, neurofibromatosis, metabolic storage disorders, familial interstitial lung disease); radiation induced fibrosis; chronic obstructive pulmonary disease (COPD); scleroderma; bleomycin induced pulmonary fibrosis; chronic asthma; silicosis; asbestos induced pulmonary fibrosis; acute respiratory distress syndrome (ARDS); kidney fibrosis; polycystic disease (PKD), autosomal dominant polycystic kidney disease (ADPKD), tubulointerstitium fibrosis; glomerular nephritis; focal segmental glomerular sclerosis; IgA nephropathy, membranous nephropathy; hypertension; Alport; gut fibrosis; liver fibrosis; cirrhosis; alcohol induced liver fibrosis; toxic/drug induced liver fibrosis; hemochromatosis; nonalcoholic steatohepatitis (NASH); biliary duct injury; primary biliary cirrhosis; infection induced liver fibrosis; viral induced liver fibrosis; and autoimmune hepatitis; corneal scarring; hypertrophic scarring; Dupuytren disease, keloids, cutaneous fibrosis; cutaneous scleroderma; systemic sclerosis, spinal cord injury/fibrosis; myelofibrosis; vascular restenosis; atherosclerosis; arteriosclerosis; Wegener's granulomatosis; Peyronie's disease, or chronic lymphocytic. More particularly, the term refers to idiopathic pulmonary fibrosis (IPF), IgA Nephropathy, Membranous Nephropathy, focal segmental glomerulo sclerosis , autosomal dominant polycystic kidney disease (ADPKD), and/or nonalcoholic steatohepatitis (NASH). Most particularly, the term refers to idiopathic pulmonary fibrosis (IPF), IgA Nephropathy, autosomal dominant polycystic kidney disease (ADPKD), and/or nonalcoholic steatohepatitis (NASH).

In one embodiment, the present invention provides compounds of the invention or pharmaceutical compositions comprising a compound of the invention, for use in the prophylaxis and/or treatment of viral infection. In particular, the term refers to influenza or flu.

In another embodiment, the present invention provides the use of compounds of the invention or pharmaceutical compositions comprising a compound of the invention in the manufacture of a medicament for the prophylaxis and/or treatment of viral infection. In particular, the term refers to influenza or flu.

In additional method of treatment aspects, this invention provides methods of prophylaxis and/or treatment of a mammal afflicted with viral infection, which methods comprise the administration of an effective amount of a compound of the invention or one or more of the pharmaceutical compositions herein described for the treatment or prophylaxis of said condition.

In one embodiment, the present invention provides pharmaceutical compositions comprising a compound of the invention, and another therapeutic agent. In a particular embodiment, the other therapeutic agent is a viral infection treatment agent.

In a particular embodiment, the present invention provides compounds of the invention or pharmaceutical compositions comprising a compound of the invention, for use in the prophylaxis and/or treatment of diseases involving degradation of cartilage and/or disruption of cartilage homeostasis. In a particular embodiment, the diseases involving degradation of cartilage and/or disruption of cartilage homeostasis is selected from osteoarthritis, psoriatic arthritis, juvenile rheumatoid arthritis, gouty arthritis, septic or infectious arthritis, reactive arthritis, reflex sympathetic dystrophy, algodystrophy, achondroplasia, Paget's disease, Tietze syndrome or costal chondritis, fibromyalgia, osteochondritis, neurogenic or neuropathic arthritis, arthropathy, sarcoidosis, amylosis, hydarthrosis, periodical disease, rheumatoid spondylitis, endemic forms of arthritis like osteoarthritis deformans endemica, Mseleni disease and Handigodu disease; degeneration resulting from fibromyalgia, systemic lupus erythematosus, scleroderma and ankylosing spondylitis. More particularly, the diseases involving degradation of cartilage and/or disruption of cartilage homeostasis is osteoarthritis (OA).

In another embodiment, the present invention provides the use of compounds of the invention or pharmaceutical compositions comprising a compound of the invention in the manufacture of a medicament for the prophylaxis and/or treatment of diseases involving degradation of cartilage and/or disruption of cartilage homeostasis. In a particular embodiment, the diseases involving degradation of cartilage and/or disruption of cartilage homeostasis is selected from osteoarthritis, psoriatic arthritis, juvenile rheumatoid arthritis, gouty arthritis, septic or infectious arthritis, reactive arthritis, reflex sympathetic dystrophy, algodystrophy, achondroplasia, Paget's disease, Tietze syndrome or costal chondritis, fibromyalgia, osteochondritis, neurogenic or neuropathic arthritis, arthropathy, sarcoidosis, amylosis, hydarthrosis, periodical disease, rheumatoid spondylitis, endemic forms of arthritis like osteoarthritis deformans endemica, Mseleni disease and Handigodu disease; degeneration resulting from fibromyalgia, systemic lupus erythematosus, scleroderma and ankylosing spondylitis. More particularly, the diseases involving degradation of cartilage and/or disruption of cartilage homeostasis is osteoarthritis (OA).

In additional method of treatment aspects, this invention provides methods of prophylaxis and/or treatment of a mammal afflicted with diseases involving degradation of cartilage and/or disruption of cartilage homeostasis, which methods comprise the administration of an effective amount of a compound of the invention or one or more of the pharmaceutical compositions herein described for the treatment or prophylaxis of said condition. diseases involving degradation of cartilage and/or disruption of cartilage homeostasis. In a particular embodiment, the diseases involving degradation of cartilage and/or disruption of cartilage homeostasis is selected from osteoarthritis, psoriatic arthritis, juvenile rheumatoid arthritis, gouty arthritis, septic or infectious arthritis, reactive arthritis, reflex sympathetic dystrophy, algodystrophy, achondroplasia, Paget's disease, Tietze syndrome or costal chondritis, fibromyalgia, osteochondritis, neurogenic or neuropathic arthritis, arthropathy, sarcoidosis, amylosis, hydarthrosis, periodical disease, rheumatoid spondylitis, endemic forms of arthritis like osteoarthritis deformans endemica, Mseleni disease and Handigodu disease; degeneration resulting from fibromyalgia, systemic lupus erythematosus, scleroderma and ankylosing spondylitis. More particularly, the diseases involving degradation of cartilage and/or disruption of cartilage homeostasis is osteoarthritis (OA).

In one embodiment, the present invention provides pharmaceutical compositions comprising a compound of the invention, and another therapeutic agent. In a particular embodiment, the other therapeutic agent is an agent for the treatment of diseases involving degradation of cartilage and/or disruption of cartilage homeostasis, which methods comprise the administration of an effective amount of a compound of the invention or one or more of the pharmaceutical compositions herein described for the treatment or prophylaxis of said condition. In a particular embodiment, the diseases involving degradation of cartilage and/or disruption of cartilage homeostasis is selected from osteoarthritis, psoriatic arthritis, juvenile rheumatoid arthritis, gouty arthritis, septic or infectious arthritis, reactive arthritis, reflex sympathetic dystrophy, algodystrophy, achondroplasia, Paget's disease, Tietze syndrome or costal chondritis, fibromyalgia, osteochondritis, neurogenic or neuropathic arthritis, arthropathy, sarcoidosis, amylosis, hydarthrosis, periodical disease, rheumatoid spondylitis, endemic forms of arthritis like osteoarthritis deformans endemica, Mseleni disease and Handigodu disease; degeneration resulting from fibromyalgia, systemic lupus erythematosus, scleroderma and ankylosing spondylitis. More particularly, the diseases involving degradation of cartilage and/or disruption of cartilage homeostasis is osteoarthritis (OA).

For the prophylaxis and/or treatment of long-term conditions, such as degenerative conditions, the regimen for treatment usually stretches over many months or years so oral dosing is preferred for patient convenience and tolerance. With oral dosing, one to four (1-4) regular doses daily, especially one to three (1-3) regular doses daily, typically one to two (1-2) regular doses daily, and most typically one (1) regular dose daily are representative regimens. Alternatively, for long lasting effect drugs, with oral dosing, once every other week, once weekly, and once a day are representative regimens. In particular, dosage regimen can be every 1-14 days, more particularly 1-10 days, even more particularly 1-7 days, and most particularly 1-3 days.

Using these dosing patterns, each dose provides from about 1 to about 1000 mg of a compound of the invention, with particular doses each providing from about 10 to about 500 mg and especially about 30 to about 250 mg.

Transdermal doses are generally selected to provide similar or lower blood levels than are achieved using injection doses.

Injection dose levels range from about 0.1 mg/kg/h to at least 10 mg/kg/h, all for from about 1 to about 120 h and especially 24 to 96 h. A preloading bolus of from about 0.1 mg/kg to about 10 mg/kg or more may also be administered to achieve adequate steady state levels. The maximum total dose is not expected to exceed about 1 g/day for a 40 to 80 kg human patient.

When used to prevent the onset of a condition, a compound of the invention will be administered to a patient at risk for developing the condition, typically on the advice and under the supervision of a physician, at the dosage levels described above. Patients at risk for developing a particular condition generally include those that have a family history of the condition, or those who have been identified by genetic testing or screening to be particularly susceptible to developing the condition.

A compound of the invention can be administered as the sole active agent or it can be administered in combination with other therapeutic agents, including other compound of the inventions that demonstrate the same or a similar therapeutic activity and that are determined to be safe and efficacious for such combined administration. In a specific embodiment, co-administration of two (or more) agents allows for significantly lower doses of each to be used, thereby reducing the side effects seen.

In one embodiment, a compound of the invention or a pharmaceutical composition comprising a compound of the invention is administered as a medicament. In a specific embodiment, said pharmaceutical composition additionally comprises a further active ingredient.

In one embodiment, a compound of the invention is co-administered with another therapeutic agent for the treatment and/or prophylaxis of a disease involving inflammation, particular agents include, but are not limited to, immunoregulatory agents e.g. azathioprine, corticosteroids (e.g. prednisolone or dexamethasone), cyclophosphamide, cyclosporin A, tacrolimus, mycophenolate, mofetil, muromonab-CD3 (OKT3, e.g. Orthocolone®), ATG, aspirin, acetaminophen, ibuprofen, naproxen, and piroxicam.

In one embodiment, a compound of the invention is co-administered with another therapeutic agent for the treatment and/or prophylaxis of arthritis (e.g. rheumatoid arthritis), particular agents include but are not limited to analgesics, non-steroidal anti-inflammatory drugs (NSAIDS), steroids, synthetic DMARDS (for example but without limitation methotrexate, leflunomide, sulfasalazine, auranofin, sodium aurothiomalate, penicillamine, chloroquine, hydroxychloroquine, azathioprine, tofacitinib, baricitinib, fostamatinib, and cyclosporin), and biological DMARDS (for example but without limitation infliximab, etanercept, adalimumab, rituximab, and abatacept).

In one embodiment, a compound of the invention is co-administered with another therapeutic agent for the treatment and/or prophylaxis of proliferative disorders, particular agents include but are not limited to: methotrexate, leukovorin, adriamycin, prednisone, bleomycin, cyclophosphamide, 5-fluorouracil, paclitaxel, docetaxel, vincristine, vinblastine, vinorelbine, doxorubicin, tamoxifen, toremifene, megestrol acetate, anastrozole, goserelin, anti-HER2 monoclonal antibody (e.g. Herceptin™), capecitabine, raloxifene hydrochloride, EGFR inhibitors (e.g. lressa®, Tarceva™, Erbitux™), VEGF inhibitors (e.g. Avastin™), proteasome inhibitors (e.g. Velcade™), Glivec® and hsp90 inhibitors (e.g. 17-AAG). Additionally, the compound of the invention according to Formula I may be administered in combination with other therapies including, but not limited to, radiotherapy or surgery. In a specific embodiment the proliferative disorder is selected from cancer, myeloproliferative disease or leukemia.

In one embodiment, a compound of the invention is co-administered with another therapeutic agent for the treatment and/or prophylaxis of autoimmune diseases, particular agents include but are not limited to: glucocorticoids, cytostatic agents (e.g. purine analogs), alkylating agents, (e.g. nitrogen mustards (cyclophosphamide), nitrosoureas, platinum compound of the inventions, and others), antimetabolites (e.g. methotrexate, azathioprine and mercaptopurine), cytotoxic antibiotics (e.g. dactinomycin anthracyclines, mitomycin C, bleomycin, and mithramycin), antibodies (e.g. anti-CD20, anti-CD25 or anti-CD3 (OTK3) monoclonal antibodies, Atgam® and Thymoglobuline®), cyclosporin, tacrolimus, rapamycin (sirolimus), interferons (e.g. IFN-β), TNF binding proteins (e.g. infliximab, etanercept, or adalimumab), mycophenolate, fingolimod and myriocin.

In one embodiment, a compound of the invention is co-administered with another therapeutic agent for the treatment and/or prophylaxis of transplant rejection, particular agents include but are not limited to: calcineurin inhibitors (e.g. cyclosporin or tacrolimus (FK506)), mTOR inhibitors (e.g. sirolimus, everolimus), anti-proliferative s (e.g. azathioprine, mycophenolic acid), corticosteroids (e.g. prednisolone, hydrocortisone), antibodies (e.g. monoclonal anti-IL-2Rα receptor antibodies, basiliximab, daclizumab), polyclonal anti-T-cell antibodies (e.g. anti-thymocyte globulin (ATG), anti-lymphocyte globulin (ALG)).

In one embodiment, a compound of the invention is co-administered with another therapeutic agent for the treatment and/or prophylaxis of asthma and/or rhinitis and/or COPD, particular agents include but are not limited to: beta2-adrenoceptor agonists (e.g. salbutamol, levalbuterol, terbutaline and bitolterol), epinephrine (inhaled or tablets), anticholinergics (e.g. ipratropium bromide), glucocorticoids (oral or inhaled). Long-acting β2-agonists (e.g. salmeterol, formoterol, bambuterol, and sustained-release oral albuterol), combinations of inhaled steroids and long-acting bronchodilators (e.g. fluticasone/salmeterol, budesonide/formoterol), leukotriene antagonists and synthesis inhibitors (e.g. montelukast, zafirlukast and zileuton), inhibitors of mediator release (e.g. cromoglycate and ketotifen), biological regulators of IgE response (e.g. omalizumab), antihistamines (e.g. ceterizine, cinnarizine, fexofenadine) and vasoconstrictors (e.g. oxymethazoline, xylomethazoline, nafazoline and tramazoline).

Additionally, a compound of the invention may be administered in combination with emergency therapies for asthma and/or COPD, such therapies include oxygen or heliox administration, nebulized salbutamol or terbutaline (optionally combined with an anticholinergic (e.g. ipratropium), systemic steroids (oral or intravenous, e.g. prednisone, prednisolone, methylprednisolone, dexamethasone, or hydrocortisone), intravenous salbutamol, non-specific beta-agonists, injected or inhaled (e.g. epinephrine, isoetharine, isoproterenol, metaproterenol), anticholinergics (IV or nebulized, e.g. glycopyrrolate, atropine, ipratropium), methylxanthines (theophylline, aminophylline, bamiphylline), inhalation anesthetics that have a bronchodilatory effect (e.g. isoflurane, halothane, enflurane), ketamine and intravenous magnesium sulfate.

In one embodiment, a compound of the invention is co-administered with another therapeutic agent for the treatment and/or prophylaxis of inflammatory bowel disease (IBD), particular agents include but are not limited to: glucocorticoids (e.g. prednisone, budesonide) synthetic disease modifying, immunomodulatory agents (e.g. methotrexate, leflunomide, sulfasalazine, mesalazine, azathioprine, 6-mercaptopurine and cyclosporin) and biological disease modifying, immunomodulatory agents (infliximab, adalimumab, rituximab, and abatacept).

In one embodiment, a compound of the invention is co-administered with another therapeutic agent for the treatment and/or prophylaxis of SLE, particular agents include but are not limited to: human monoclonal antibodies (belimumab (Benlysta)), Disease-modifying antirheumatic drugs (DMARDs) such as antimalarials (e.g. plaquenil, hydroxychloroquine), immunosuppressants (e.g. methotrexate and azathioprine), cyclophosphamide and mycophenolic acid, immunosuppressive drugs and analgesics, such as nonsteroidal anti-inflammatory drugs, opiates (e.g. dextropropoxyphene and co-codamol), opioids (e.g. hydrocodone, oxycodone, MS Contin, or methadone) and the fentanyl duragesic transdermal patch.

In one embodiment, a compound of the invention is co-administered with another therapeutic agent for the treatment and/or prophylaxis of psoriasis, particular agents include but are not limited to: topical treatments such as bath solutions, moisturizers, medicated creams and ointments containing coal tar, dithranol (anthralin), corticosteroids like desoximetasone (Topicort™), fluocinonide, vitamin D3 analogues (for example, calcipotriol), argan oil and retinoids (etretinate, acitretin, tazarotene), systemic treatments such as methotrexate, cyclosporine, retinoids, tioguanine, hydroxyurea, sulfasalazine, mycophenolate mofetil, azathioprine, tacrolimus, fumaric acid esters or biologics such as Amevive™, Enbrel™, Humira™, Remicade™, Raptiva™ and ustekinumab (a IL-12 and IL-23 blocker). Additionally, a compound of the invention may be administered in combination with other therapies including, but not limited to phototherapy, or photochemotherapy (e.g. psoralen and ultraviolet A phototherapy (PUVA)).

In one embodiment, a compound of the invention is co-administered with another therapeutic agent for the treatment and/or prophylaxis of allergic reaction, particular agents include but are not limited to: antihistamines (e.g. cetirizine, diphenhydramine, fexofenadine, levocetirizine), glucocorticoids (e.g. prednisone, betamethasone, beclomethasone, dexamethasone), epinephrine, theophylline or anti-leukotrienes (e.g. montelukast or zafirlukast), anti-cholinergics and decongestants.

By co-administration is included any means of delivering two or more therapeutic agents to the patient as part of the same treatment regime, as will be apparent to the skilled person. Whilst the two or more agents may be administered simultaneously in a single formulation, i.e. as a single pharmaceutical composition, this is not essential. The agents may be administered in different formulations and at different times.

### EXPERIMENTAL SECTION

### General Methods

X-ray powder diffraction data were collected using a PANalytical Empyrean diffractometer (PANalytical, Amlelo, The Netherlands) fitted with a X'cellerator detector. The radiation used was CuKα (1.54A) and the voltage and current were set at 45 kV and 40 mA respectively. Data were collected at room temperature from 3 to 40 degrees 2-theta with a step size of 0.013 degrees 2-theta. Samples were prepared on a holder disc between Mylar® films and the diffraction profiles were recorded in the transmission mode with sample spinning

Differential Scanning Calorimetry (DSC) data was collected using a DSC Q1000 (or DSC Q2000) Differential Scanning Calorimeter (TA Instruments, Newcastle, DE, USA). Data was collected at a heating rate of 10° C./min over a temperature range of 25° C. to 250° C. Analyses were run under nitrogen and samples were loaded in standard pierced aluminium pans. Indium was used as a calibration standard.

Thermogravimetric Analyses (TGA) data was collected using a TGA Q5000 Thermogravimetric Analyser (TA Instruments, Newcastle, DE, USA). Data were collected at a heating rate of 10° C./min over a temperature range of 25° C. to 250° C. Analyses were run under nitrogen and samples were loaded in standard open aluminium pans. Nickel was used as a temperature calibration standard using the Curie point method.

Infra-red spectra were recorded using a Bruker ALPHA II infrared spectrophotometer (Bruker, Wissembourg, France). Data was collected at room temperature range in the ATR mode with resolution of 2 cm⁻¹ following the averaging of 24 scans.

Solid-state ¹³C NMR spectra were recorded at ambient temperature using a Bruker SB Avance III HD 500 spectrometer with a 4 mm CP/MAS SB VTN type probe under the following conditions:
Frequency: 125.76 MHz,
Spectral width: 37 kHz,
Magic angle spinning rate: 10 kHz,
Pulse program: Cross Polarization with SPINAL64 decoupling
Recycle delay: 10 s,
Acquisition time: 46 ms,
Contact time: 4 ms,
   Number of scans: 4096.
   A 5 Hz line-broadening was applied prior to FT. Spectra thereby obtained were referenced relative to a sample of adamantane (the high frequency peak of adamantane was set to 38.5 ppm).

Solid-state ¹⁵N NMR spectra were recorded at ambient temperature using a Bruker SB Avance III HD 500 spectrometer with a 4 mm CP/MAS SB VTN type probe under the following conditions:
Frequency: 50.68 MHz,
Spectral width: 25 kHz,
Magic angle spinning rate: 5 kHz,
Pulse program: Cross Polarization with SPINAL64 decoupling
Recycle delay: 30 s,
Acquisition time: 40 ms,
Contact time: 4 ms,
Number of scans: 8192.

A 5 Hz line-broadening was applied prior to Fourier transformation (FT). Spectra obtained were referenced relative to a sample of ¹⁵N-doped glycine (the frequency was taking between two peaks of glycine and was set to 33.4 ppm in the reference frame where ammonia corresponds to 0 ppm±0.2 ppm.

Water sorption/desorption profiles were recorded on a Dynamic Vapor Sorption System, DVS (Surface Measurement Systems Ltd., London, UK). Approximately 5 to 10 mg of sample substance was placed in the pan of the DVS instrument working at 25° C. under controlled humidity. The mass variation was recorded as a function of relative humidity following the program detailed hereafter:
the sample was equilibrated at 50 per cent RH until the mass variation was less than 0.002% per min within a limit of time of 6 h.
the relative humidity was increased from 50 per cent RH to 90 per cent RH at a rate of 10% per h.
the sample was equilibrated at 90 per cent RH until the mass variation was less than 0.002% per min within a limit of time of 6 h.
the relative humidity was decreased from 90 per cent RH to 0 per cent RH at a rate of 10% per h.
the sample was equilibrated at 0 per cent RH until the mass variation is less than 0.002% per min within a limit of time of 6 h.
the relative humidity was increased from 0 per cent RH to 50 per cent RH at a rate of 10% per h.

Abbreviations

| | | | |
|---|---|---|---|
| % wt | Percentage by weight | DIPE | Diisopropyl ether |
| μCT | micro-computed tomography | DMD | Duchenne muscular dystrophy |
| μg | microgram | DMSO | Dimethyl sulfoxide |
| ALP | Alkaline phosphatase | DSC | Differential scanning calorimetry |
| ALT | alanine aminotransferase | EDTA | Ethylenediaminetetraacetic acid |
| AST | aspartate aminotransferase | ELF | Enhanced Liver Fibrosis |
| AUC | area under the curve | FFPE | Formalin-Fixed Paraffin-Embedded |
| Av g/g | average force output/g of body | FITC- | fluorescein isothiocyanate - |
| BW | weight | WGA | Triticum vulgaris Lectin |
| b.i.d. | bis in diem - twice daily dosing | H&E | Hematoxylin and Eosin |
| BSA | Bovine serum albumin | h | hour |
| CDHFD | choline-deficient, L-amino acid- | IPA | Isopropyl alcohol |
| | defined, high-fat diet | IR | Infra-red spectroscopy |
| Cl/F | clearance | MCH | Methyl cyclohexane |
| Cmax | peak exposure | mg | milligram |
| CV % | coefficient of variation | MIK | Methyl isobutyl ketone |
| DCM | Dichloromethane | min | minute |
| mL | milliliters | q.d. | quo die - once daily dosing |
| MSM | Main starting material | QC | Quality control |
| MTBE | Methyl tert-butyl ether | QC | quality controlled |
| MTHF | 2-Methyl Tetrahydrofuran | RH | Relative humidity |
| N,N- | N,N-diisopropylethylamine | SD | standard deviation |
| DIPEA | | SEM | standard error of the mean |
| NEFA | Non-Esterified Fatty Acids | T½ | half life |
| NITEGE | Aggrecan monoclonal antibody to | TGA | Thermogravimetric analysis |
| | C-terminal neoepitope | THF | tetrahydrofuran |
| NMR | Nuclear magnetic resonance | Tmax | Time to peak exposure |
| p.o | per os - orally dosed | Vol/w | Volume equivalent mass |
| PBS | Phosphate buffered saline | XRD | X-Ray diffraction |
| PK | pharmacokinetic | XRPD | X-Ray powder diffraction |

### Example 1. Synthetic Preparation of the Solid Forms of the Invention

### 1.1. Preparation of amorphous (5S)-cyclopropyl-5-[3-[(3S)-4-(3,5-difluorophenyl)-3-methyl-piperazin-1-yl]-3-oxo-propyl]imidazolidine-2,4-dione

Amorphous material may be obtained following the procedure for the preparation of Cpd 1 described in WO 2016/102347 or alternatively by heating polymorphic form I to 100° C. under vacuum.

The amorphic form was characterized by XRPD as shown on FIG. 18.

The amorphic form was characterized by DSC substantially as shown in FIG. 20.

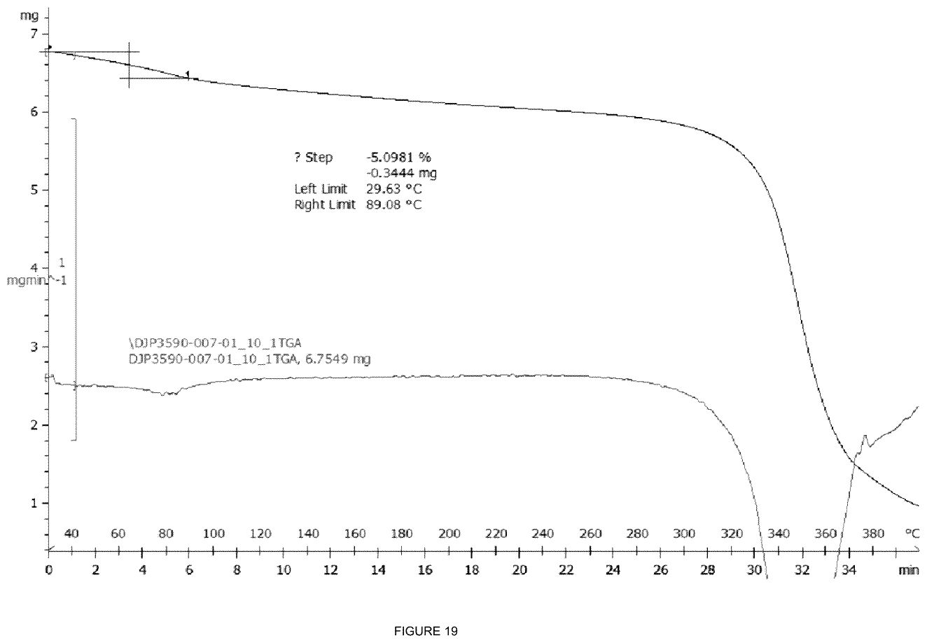

The amorphic form was characterized by thermogravimetric analysis (TGA) substantially as shown on FIG. 19.

### 1.2. Preparation of polymorphic form I of (5S)-cyclopropyl-5-[3-[(3S)-4-(3,5-difluorophenyl)-3-methyl-piperazin-1-yl]-3-oxo-propyl]imidazolidine-2,4-dione

### 1.2.1. Form I—Protocol 1

(5S)-cyclopropyl-5-[3-[(3S)-4-(3,5-difluorophenyl)-3-methyl -piperazin-1-yl]-3-oxo-propyl]imidazolidine-2,4-dione and IPA (3.93 kg/kg MSM) are loaded in a reactor at 20° C. The suspension is heated at 40° C. resulting in the formation of a solution that is maintained at this temperature during 15 min. The said solution is then clarified by filtration on a PALL filter.

A first load of water (5 kg/kg MSM) is added at 40° C. in 30 min. The solution is cooled to 35° C. in 15 min (slope=−0.3° C./min) then seeded with crystalline (5S)-cyclopropyl-5-[3-[(3S)-4-(3,5-difluorophenyl)-3-methyl-piperazin-1-yl]-3-oxo-propyl]imidazolidine-2,4-dione. After 15 min, the suspension is cooled to 5° C. in 100 min (slope=−0.3° C./min).

A second load of water (2.5 kg/kg MSM) is added in 15 min at 5° C. and the medium is maintained under stirring at 5° C. for at least 10 h before filtration through a 20-μm filter.

The cake is washed twice with previously cooled (5° C.) water (2×2.50 kg/kg MSM). The solid obtained is dried in ventilated oven at 40° C.

Polymorphic Form I of (5S)-cyclopropyl-5-[3-[(3S)-4-(3,5-difluorophenyl)-3-methyl-piperazin-1-yl]-3-oxo-propyl]imidazolidine-2,4-dione is obtained with an 81% yield.

Polymorphic form I was characterized by XRPD (FIG. 1).

Polymorphic form I was characterized by DSC substantially as shown in FIG. 3 and displaying an endothermic transition around 75-85° C. (onset), and more particularly at about 80° C. when Form I is heated in an pierced aluminium pan when heated from about 25° C. at a rate of 10° C./min.

Polymorphic form I was characterized by thermogravimetric analysis (TGA) substantially as shown on FIG. 5, showing a weight loss in a range of about 8%. This weight loss was determined to be water via Karl Fischer (KF) analysis. KF analysis shows that the water content can be about 8%, corresponding to a dihydrate.

Polymorphic form I was characterized by Infra-red spectroscopy (IR) substantially as shown on FIG. 7, showing peaks at the following positions: 3488.5, 1751.3, 1719.0, 1598.4, 1586.0, 1484.5, 1458.0, 1439.3, 1410.5, 1252.7, 1199.9, 1113.1, 1051.7,1026.5, 992.2, 835.2, 807.5, and 675.7 cm⁻¹.

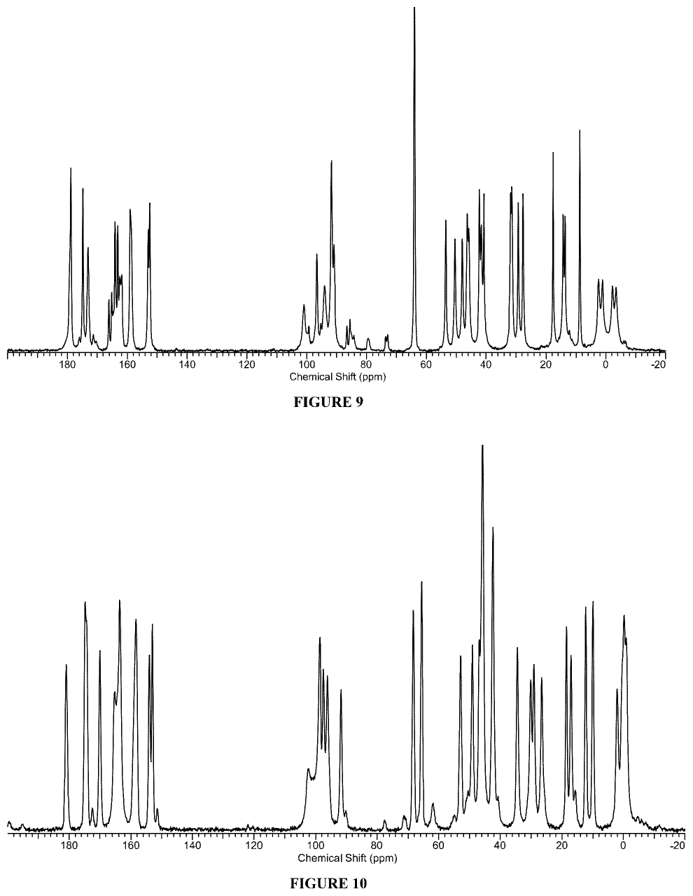

Polymorphic form I was characterized by ¹³C solid state NMR substantially as shown on FIG. 9, showing peaks at the following positions: −3.7, −2.2, 1.2, 2.5, 8.7, 13.7, 14.3, 27.7, 29.2, 31.4, 31.8, 40.7, 42.2, 45.8, 46.3, 48.0, 50.4, 53.5, 64.1, 90.9, 91.7, 94.0, 96.6, 100.9, 152.4, 153.0, 159.1, 163.2, 164.1, 165.1, 166.1, 173.0, 174.8, 178.8 ppm.

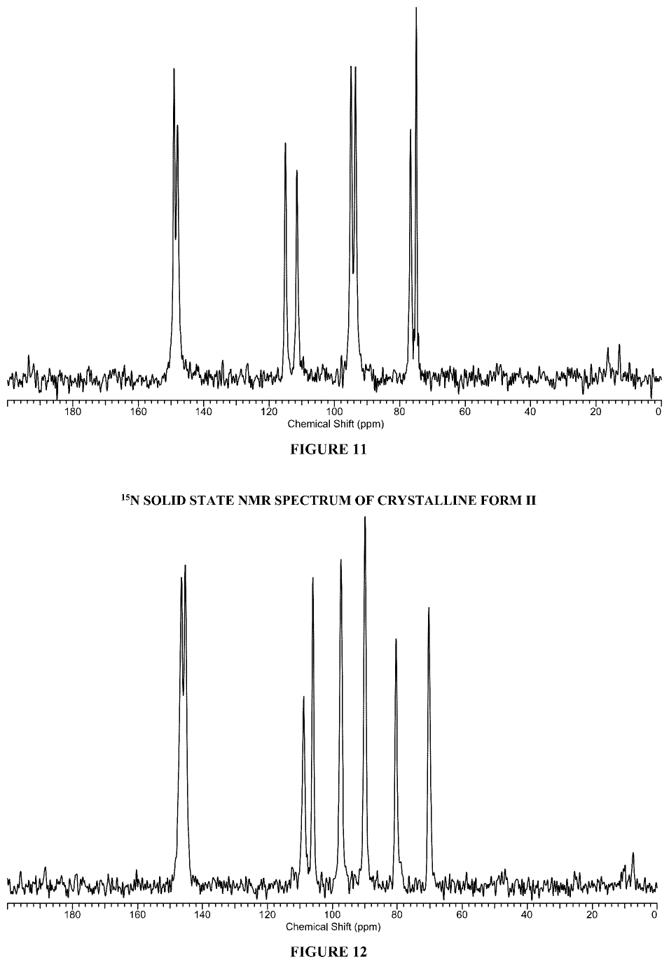

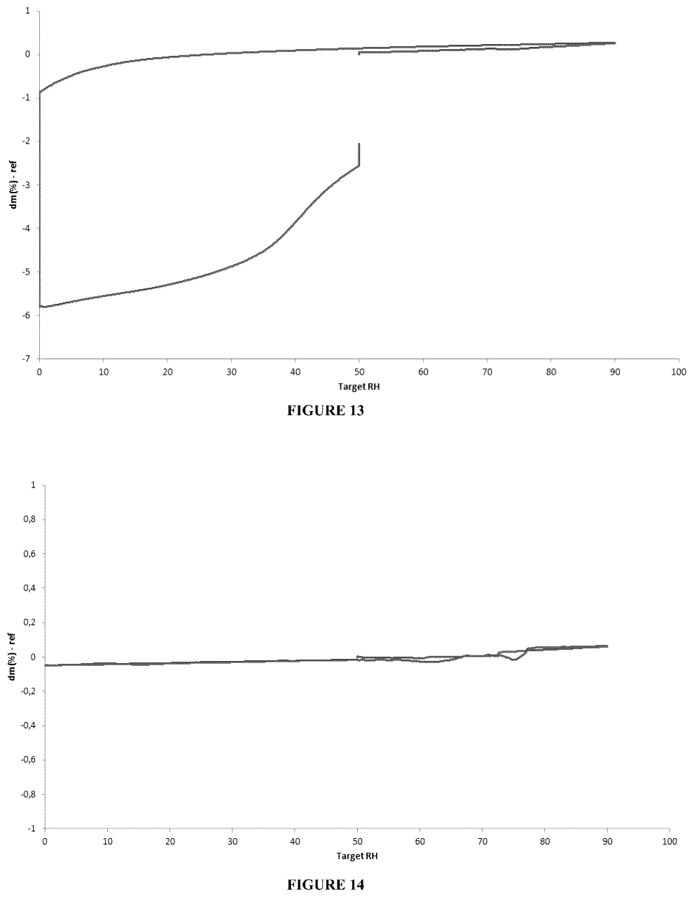

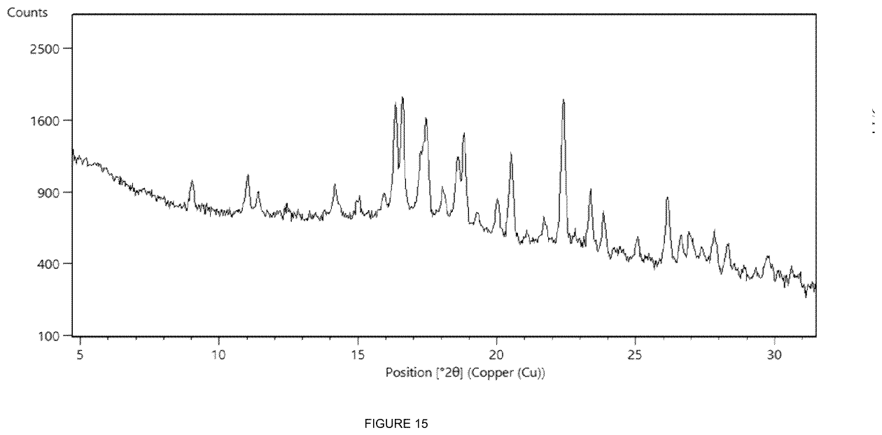

Polymorphic form I was characterized by ¹⁵N solid state NMR substantially as shown on FIG. 11, showing peaks at the following positions: 75.0, 76.8, 93.6, 94.9, 111.5, 115.1, 148.0, 149.1 ppm

### 1.2.2. Form I—Protocol 2

Amorphous Cpd 1 (˜50 mg) was dissolved in acetone and subjected to stirring for 30 min in a glass vial with caps to give clear solutions. Water (0.5 mL to 1.5 mL) was then added under stirring resulting in the precipitation of Cpd 1 as Form I.

### 1.3. Preparation of Polymorphic Form II of (5S)-cyclopropyl-5-[3-[(3S)-4-(3,5 difluorophenyl)-3-methyl-piperazin-1-yl]-3-oxo-propyl]imidazolidine-2,4- dione

### 1.3.1. Form II—Protocol 1

(5S)-cyclopropyl-5-[3-[(3S)-4-(3,5-difluorophenyl)-3-methyl-piperazin-1-yl]-3-oxo-propyllimidazolidine-2,4-dione and IPA (3.93 kg/kg MSM) are loaded in a reactor at 20° C. The suspension is heated at 40° C. resulting in the formation of a solution that is maintained at this temperature during 30 min. The said solution is then clarified by filtration on a PALL filter and seeded by crystalline (5S)-cyclopropyl-5-[(3S)-4-(3,5-difluorophenyl)-3-methyl-piperazin-1-yl]-3-oxo-propyl]imidazolidine-2,4-dione (0.01 kg/kg MSM).

After 15 min, the medium is cooled to 25° C. in 150 min (slope=−0.1° C./min) and maintained at this temperature during 60 min.

A methyl cyclohexane load (8.18 kg/kg MSM) is added in 90 min at 25° C. The mixture is maintained at this temperature during 60 min and is then cooled to 5° C. in 200 min (slope=−0.1° C./min) and maintained under stirring at this temperature during 12 h before filtration through a 20-μm filter.

The cake is washed with previously cooled (5° C.) MCH (2.40 kg/kg MSM). The solid obtained is dried under vacuum at 40-50° C.

Polymorphic form II of (5S)-cyclopropyl-5-[3-[(3S)-4-(3,5-difluorophenyl)-3-methyl-piperazin-1-yl]-3-oxo-propyl]imidazolidine-2,4-dione is obtained with a 65.6% yield.

Residual solvents: MCH (0.8%), IPA (<0.1%)

### 1.4. Form II—Protocol 2

(5S)-cyclopropyl-5-[3-[(3S)-4-(3,5-difluorophenyl)-3-methyl-piperazin-1-yl]-3-oxo-propyl]imidazolidine-2,4-dione and methyl isobutyl ketone (5 kg/kg MSM) are loaded in a reactor at 20° C. The suspension is heated at 60° C. resulting in the formation of a solution that is maintained at this temperature during 15 min. The said solution is then clarified by filtration on a PALL filter. A first load of DIPE (5 kg/kg MSM) is added at 60° C. in 45 min. The solution is then seeded by crystalline (5S)-cyclopropyl-5-[3-[(3S)-4-(3,5-difluorophenyl)-3-methyl-piperazin-1-yl]-3-oxo-propyl]imidazolidine-2,4-dione (0.025 kg/kg MSM). After 15 min, a further load of DIPE (5 kg/kg MSM) is added in 30 min. The medium is cooled at 0° C. according to the following protocol:
Cooling at 50° C. in 30 min (slope=−0.3° C./min), 60 min contact time at 50° C.,
Cooling at 40° C. in 30 min (slope=−0.3° C./min), 120 min contact time at 40° C.,
Cooling at 30° C. in 30 min (slope=−0.3° C./min), 120 min contact time at 30° C.,
Cooling at 20° C. in 30 min (slope=−0.3° C./min), 120 min contact time at 20° C.,
Cooling at 10° C. in 30 min (slope=−0.3° C./min), 120 min contact time at 10° C.,
Cooling at 0° C. in 30 min (slope=−0.3° C./min), 120 min contact time at 0° C.

The medium is filtered through a 20-μm filter.

The cake is washed with previously cooled (0° C.) DIPE (5 kg/kg MSM). The solid obtained is dried under vacuum at 40-50° C.

Polymorphic form II was characterized by XRPD as shown on FIG. 2.

Polymorphic form II was characterized by DSC substantially as shown in FIG. 4 and displaying an endothermic transition around 155-162° C. (onset), and more particularly at about 159° C. when Form II is heated in an pierced aluminium pan when heated from about 25° C. at a rate of 10° C./min.

Polymorphic form II was characterized by thermogravimetric analysis (TGA) substantially as shown on FIG. 6.

Polymorphic form II was characterized by infra-red spectroscopy (IR) substantially as shown on FIG. 7, showing peaks at the following positions: 1717.8, 1624.6, 1584.8, 1447.1, 1193.0, 1556.0, 1111.4, 1022.8, 987.4, 823.3, 765.1, and 672.7cm⁻¹.

Polymorphic form II was characterized by ¹³C solid state NMR substantially as shown on FIG. 9, showing peaks at the following positions: −0.9, 0.3, 2.1, 10.0, 12.3, 17.0, 29.1, 30.1, 34.4, 42.4, 45.7, 46.8, 49.1, 52.9, 65.6, 68.3, 91.7, 96.1, 97.5, 98.6, 102.3, 153.0, 153.9, 158.3, 163.6, 165.1, 170.0, 174.8, 180.9 ppm

Polymorphic form II was characterized by ¹⁵N solid state NMR substantially as shown on FIG. 11, showing peaks at the following positions: 70.3, 80.4, 89.9, 97.4, 106.0, 108.9, 145.3, 146.5 ppm.

### 1.4.1. Form II—Protocol 3

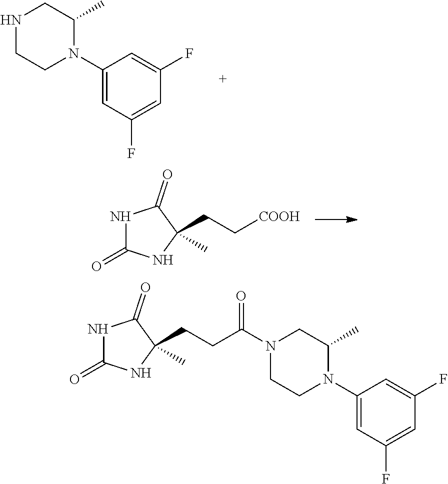

To a suspension of (4R)-4-Methyl-2,5-dioxo-4-imidazolidinepropanoic acid (reference mass 1 wt, CAS 1957994-69-2) in methyl isobutyl ketone (7.8 equiv wt/wt) under nitrogen at ambient temperature is added under stirring a first batch of N,N-DIPEA (0.6 equiv wt/wt) over 60 min, followed by a second batch of N,N-DIPEA (1.5 equiv wt/wt) over 60 min.

(2S)-1-(3,5-Difluorophenyl)-2-methylpiperazine (2.0 equiv wt/wt, CAS 845740-76-3) is then added in about 70 min maintaining the temperature between 20 and 25° C., followed by propanephosphonic acid anhydride (50%) in methyl THF (3.6 equiv wt/wt) maintaining the temperature in the range 20-25° C. in about 2.5 h.

The reaction mixture was kept under stirring at 20-25° C. for 45 min and then the batch temperature was increased to 38-42° C. in 1 h and kept for 2 h and 15 min before cooling to 20-30° C.

Purified water (4.9 equiv wt/wt) was dosed into the reaction mixture over 70 min maintaining the temperature in the range 20-30° C. and kept under stirring at that temperature for an additional 15 min. Stirring was stopped and the phases were separated. To the organic layer was added a solution of 32% aqueous solution hydrochloric acid (0.3 equiv wt/wt) in purified water (4.9 equiv wt/wt), in 30 min keeping batch during the addition at 20-25° C.

The batch was kept under stirring, then the stirrer was stopped, the phases were separated, and a 5% wt ammonium chloride aqueous solution (4.9 equiv wt/wt) was added, and the batch was stirred at 20-25° C. Then the stirrer was stopped, the phases were separated. Again, 5% wt ammonium chloride aqueous solution (4.9 equiv wt/wt) was added, and the batch was stirred at 20-25° C. Then the stirrer was stopped, the phases were separated. The organic phase was washed again with purified water (4.9 equiv wt/wt) and the organic phase was separated and distilled, first at atmospheric pressure then under vacuum, till the internal temperature reached higher values than 101° C.

The residue was then diluted in methyl isobutyl ketone (1.4 equiv wt/wt) and heated to about 60° C. for about 17 min and then cooled to 20-25° C. The batch was filtered through a 0.5 μm cartridge, the cartridge was rinsed with methyl isobutyl ketone (1.4 equiv wt/wt).

The resulting solution was heated to 58-62° C. and diisopropyl ether (11.7 equiv wt/wt) was added maintaining the batch at the temperature of 58-62° C. over 25 min. The solution was seeded, 3 other portions of diisopropyl ether (2.6 equiv wt/wt, 2.6 equiv wt/wt and 8.9 equiv wt/wt) were added before cooling the resulting suspension to 0-5° C. over 14 h. The resulting suspension was left at that temperature for about 2 h, then the solid was separated by centrifugation, washed with pre-cooled diisopropyl ether (8.9 equiv wt/wt) and dried at 48° C. for 20 h to afford the title compound.

### 1.4.2. Form II—Protocol 4

Amorphous Cpd 1 (˜50 mg) was dissolved in methanol or ethanol and subjected to stirring for 30 min in a glass vial with caps to give clear solutions. Water (0.5 mL to 1.5 mL) was then added under stirring resulting in the precipitation of Cpd 1 as Form II.

### 1.4.3. Form II—Protocol 5

Amorphous Cpd 1 (498 mg) and 5 mL MTBE were stirred until the obtention of a clear solution, and then for a further 3 h resulting in the precipitation of Cpd 1 as Form II, which was separated by filtration and finally dried.

### 1.5. Preparation of Polymorphic Form III of (5S)-cyclopropyl-5-[3-[(3S)-4-(3,5-difluorophenyl)-3-methyl-piperazin-1-yl]-3-oxo-propyl]imidazolidine-2,4-dione

Amorphous material (200 mg) is slurried in MTBE (400 μL) at room temperature. To speed up crystallization of form 3, the mixture may be seeded with form 3 after 2 h of stirring. After 3 days, the solid is dried at free air and analyzed by XPRD, TGA and DSC.

Polymorphic form III was characterized by XRPD as shown on FIG. 15.

Polymorphic form III was characterized by DSC substantially as shown in FIG. 16 and displaying a transition around 111-170° C. (onset), and more particularly at about 140° C. when Form III is heated in an pierced aluminium pan when heated from about 25° C. at a rate of 10° C./min.

Polymorphic form III was characterized by thermogravimetric analysis (TGA) substantially as shown on FIG. 17.

### Example 2. Stability Study of the Solid Forms of the Invention

The stability of solid forms of Cpd 1 as amorphous, form I and form II were evaluated under different conditions , which are reported below, indicating that all 3 forms are stable.

| Storage condition | Amorphous | Form I | Form II |
|---|---|---|---|
| 25° C./60% RH - 12 months | 100% | 100% | 99.3% |
| 40° C./75% RH - 6 months | 99.3 | 100% | 98.7% |

### BIOLOGICAL DATA

### Example 3. Pharmaceutical Compositions

### 3.1. PK Study in Human

Following a single 300 mg oral administration of Cpd 1 under fasted and/or fed conditions to healthy male subjects (18 subjects per group) as 30% or 50% drug load tablets of crystalline form I and crystalline form II, concentrations of Cpd 1 human plasma concentrations were determined using protein precipitation extraction with liquid chromatography with tandem mass spectrometry (LC-MS/MS) detection.

The lower limit of quantification was 1 ng/mL. In each analytical run, duplicate QC samples were analyzed along with the study samples.

Blood samples (2 mL) for determination of Cpd 1 in plasma were collected at various time points into tubes containing lithium heparin and were immediately chilled (ice bath). Within 30 min after blood collection, the plasma was separated in a refrigerated centrifuge at 4° C. for 10 min at circa 1,500 g.

Predose PK samples were collected within 15 min predose then at the following time points: 0.5 h, 1 h, 1.5 h, 2 h, 2.5 h, 3 h, 4 h, 5 h, 6 h, 8 h, 12 h, 16 h, 24 h, 48 h, 72 h. For all PK samples between 0.5 and 16 h, a window of ±5 min was allowed; for 24- to 72-h samples, a window of ±30 min was allowed.

All statistical analyses were performed using SAS® version 9.4 (or higher) (SAS Institute, Cary, NC, USA) and/or Phoenix WinNonLin (version 8 or higher) software.

All relevant data were documented using summary tables, figures, and subject data listings.

Summary tabulations displayed the number of non-missing observations, arithmetic mean, standard deviation (SD) and/or standard error (as appropriate) of the arithmetic mean, median, minimum, and maximum for continuous variables, and the number and percentage per category for categorical data. For PK data, also the number of data points above the lower limit of quantification (only applicable for concentrations), the coefficient of variation (CV %), the geometric mean, and the geometric CV % were displayed. In addition to tabulated descriptive statistics, graphical data displays were used to summarize the data. Inferential statistics were interpreted at the 2-sided 5% significance level, unless otherwise noted.

TABLE I
PK measured parameters for Form I & Form II

| | Form I | | Form II | | Form II | | Form II | |
|---|---|---|---|---|---|---|---|---|
| | 30% load tablet | | 30% load tablet | | 50% load tablet | | 50% load tablet | |
| PK Parameters | Fasted | | fasted | | fasted | | fed | |
| Tmax (h) | 4.00 | | 4.00 | | 4.00 | | 4.00 | |
| | (range 1.50-6.00) | | (range 2.00-6.00) | | (range 1.00-6.00) | | (range 1.50-8.00) | |
| Cmax (ng/mL) | 2546 | (18.3) | 3419 | (20.8) | 3568 | (22.0) | 4252 | (24.5) |
| AUC0-24 h | 30909 | (14.7) | 37666 | (17.7) | 37988 | (19.9) | 42285 | (21.3) |
| (ng · h/mL) | | | | | | | | |
| AUC0-t (ng · h/mL) | 41686 | (17.2) | 46247 | (19.0) | 46233 | (19.9) | 49374 | (24.6) |
| AUC0-∞ (ng · h/mL) | 42613 | (18.5) | 46593 | (19.5) | 46557 | (20.3) | 49540 | (24.7) |
| t½ (h) | 11.3 | (31.4) | 8.90 | (23.4) | 8.44 | (26.4) | 8.03 | (15.3) |
| CL/F (L/h) | 7.04 | (18.5) | 6.44 | (19.5) | 6.44 | (20.3) | 6.06 | (24.7) |

When tested according to this protocol, although there was no difference in median tₘₐₓ, for all tablets, an increase in plasma peak exposure (Cₘₐₓ) was observed for polymorphic form II compared to polymorphic form I (34% and 40% for the 30% and 50% load tablets under fasted condition respectively).

### Example 4. In Vivo Models

### 4.1. Protein Overload Model Nephrectomy Model

This animal model allows to assess the efficacy of the compound of the invention in renal disease, including renal fibrosis.

### 4.1.1. Materials

This study was undertaken in Balb/c mice (Charles River).

All the mice were kept under standardised conditions of 12 hr day/12 hr night light cycles, were fed with standard chow (“https://insights.envigo.com/hubfs/resources/data-sheets/2018-datasheet-0915.pdf,” n.d.) and had ad libitum access to water.

Animal were acclimatised for at least a week before commencement of procedures.

Mice underwent a left unilateral nephrectomy via a short flank incision under anaesthesia.

Sham control mice received an incision only . The mice were allowed to recover from surgery for 7 days.

### 4.1.2. Experimental Protocol

Drug treatment commenced one day prior to initiation of protein overload.

Each mouse received either Cpd 1 240 mg/kg/day b.i.d. (dosed daily at 8h00 and 15h30), or 10 mg/kg/day Lisinopril (one dose Lisinopril plus one dose of vehicle), or vehicle alone (0.5% methyl cellulose containing 2% Tween 80—two doses) by oral gavage.

Sham mice received vehicle alone. Protein overload was initiated by daily intraperitoneal injections of sterile filtered 450 mg/ml BSA solution in saline (Sigma cat A4919: low endotoxin) starting from 2 mg/g body weight on day one and increasing to 15 mg/g on day 7 until day 14 according to schedule below:

TABLE II
Protein Overload model Nephrectomy model dosing schedule

| day | 1 | 2 | 3 | 4 | 5 | 6 | 7-14 |
|---|---|---|---|---|---|---|---|
| dose/g body | 2 | 4 | 6 | 8 | 10 | 12 | 15 |
| weight (mg) | | | | | | | |
| volume (μL) | 133.3 | 266.7 | 400 | 533.3 | 666.7 | 800 | 1000 |
| (~30 g mouse) | | | | | | | |

Sham mice received equivalent volumes of saline. After the last injection, mice were housed in metabolic cages for 24 h urine collection.

The mice were then anaesthetized before sacrifice. Blood sample was collected. The remaining right kidneys were perfused with PBS (Sigma 806552)+10 mM EDTA (Invitrogen 15575-02—stock 0.5M) after which they were excised and transverse dissected.

One half was placed in formalin in preparation for paraffin blocking for use in immunohistochemistry, while the other half was patted dry and placed into liquid nitrogen for later proteomics analysis. The spleens were also removed and cut in half for FFPE and proteomics.

### 4.1.3. Statistical Analysis Method

All data were expressed as means±SEM. Results were analysed using GraphPad Prism 8. D'Agostino-Pearson omnibus normality tests were performed to assess normality for data sets >8, while a Shapiro-Wilk test was applied to data sets <8. For multiple comparisons, a one-way ANOVA with Tukey's post hoc correction (parametric) or a Kruskal-Wallis test with Dunn's post hoc test (non-parametric data) were used. ELISA standard curves were plotted using a Sigmoidal4PL fit where x was the log of the standard concentration. Relative mRNA expression levels were calculated according to the Livak equation as 2-ΔCt. A confidence interval of 0.95, p≤0.05, was applied for all statistical tests.

The following endpoints were measured:
Urine Protein concentration mg/mL,
Urine protein/mg creatinine (mg/mg)
serum protein concentration (mg/mL),
urine creatinine concentration (mg/dL)(mg/mL)
Serum creatinine concentration (mg/dL)
Serum urea concentration (mg/dL)
Versikine, NITEGE, ARGS dot blots
Immunohistochemistry: ADAMTS5, Collagen I, Collagen IV, fibronectin, CD45, complement C3, F4/80

### 4.1.4. Results

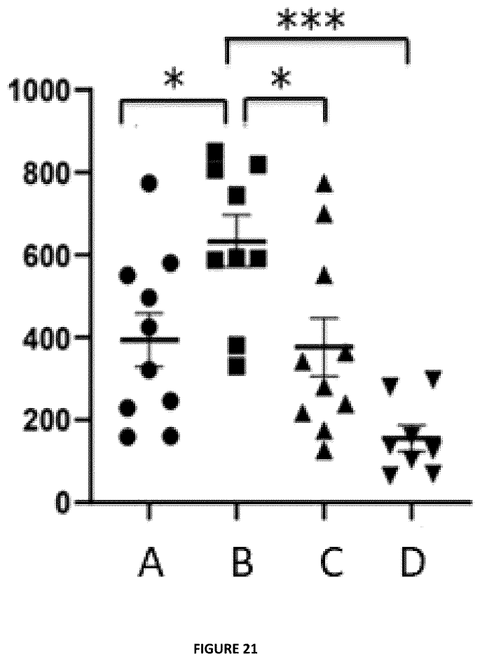

When subjected to the above-mentioned protocol, the following values were measured at day 14 (see FIG. 21). Cpd 1 showed a statistically significant improvement in proteinuria compared to the vehicle group (p<0.05, *) as shown in the tables below:

TABLE III
Individual subject proteinuria - FIG. 21

| | serum creatinine | urine creatinine | proteinuria | Protein/ |
|---|---|---|---|---|
| Grp | (mg/dL) | (mg/mL) | (mg/mL) | creatinine |
| Cpd 1 | 0.39 | 0.26 | 143.5 | 550 |
| (Group A) | 0.38 | 0.297 | 229.6 | 774 |
| | 0.3 | 0.384 | 163 | 425 |
| | 0.37 | 0.28 | 44.4 | 159 |
| | 0.43 | 0.33 | 75.2 | 229 |
| | 0.4 | 0.34 | 54.7 | 160 |
| | 0.47 | 0.38 | 187 | 497 |
| | 0.44 | 0.25 | 79.4 | 321 |
| | 0.41 | 0.25 | 145 | 581 |
| | 0.46 | 0.29 | 72.4 | 250 |
| Vehicle | 0.36 | ND | ND | ND |
| (Group B) | ND | 0.29 | 110.1 | 380 |
| | 0.39 | 0.409 | 135 | 330 |
| | 0.38 | 0.46 | 342.5 | 745 |
| | 0.46 | 0.39 | 231.3 | 588 |
| | 0.43 | 0.24 | 200 | 851 |
| | 0.41 | 0.32 | 258 | 806 |
| | 0.35 | 0.38 | 308 | 819 |
| | 0.37 | 0.36 | 213 | 591 |
| | 0.47 | 0.24 | 144 | 600 |
| lisinopril | 0.39 | 0.467 | 59.1 | 127 |
| (Group C) | 0.41 | 0.378 | 128.8 | 341 |
| | 0.34 | 0.32 | 55.8 | 174 |
| | 0.35 | 0.36 | 197.6 | 552 |
| | 0.25 | 0.3 | 71.7 | 238 |
| | 0.57 | 0.34 | 264.6 | 774 |
| | 0.57 | 0.41 | 284 | 700 |
| | 0.46 | 0.38 | 82.1 | 216 |
| | 0.55 | 0.22 | 80 | 364 |
| | 0.54 | 0.42 | 79 | 188 |
| Sham | 0.25 | 0.427 | 44.4 | 104 |
| (Group D) | 0.3 | 0.81 | 112.7 | 138 |
| | 0.25 | 0.26 | 41.8 | 160 |
| | 0.41 | 0.29 | 36.6 | 127 |
| | 0.39 | 0.73 | 50.8 | 69.5 |
| | 0.45 | 0.37 | 24.3 | 65.8 |
| | 0.42 | 0.77 | ND | ND |
| | 0.44 | 0.2 | 61 | 305 |
| | 0.44 | 0.23 | 64 | 278 |

TABLE IV
Statistical analysis proteinuria - FIG. 21

| | Cpd 1 | Vehicle | lisinopril | Sham |
|---|---|---|---|---|
| Group | (Group A) | (Group B) | (Group C) | (Group D) |
| Mean | 394.2 | 633.8 | 376.4 | 155.3 |
| Protein/creatinine | | | | |
| (mg/mL) | | | | |
| Std. Deviation | 205.3 | 188.8 | 224.2 | 88.7 |
| Std. Error of | 64.9 | 62.9 | 70.9 | 31.4 |
| Mean | | | | |
| Anova analysis | vs group B: 0.0433 | vs group C: 0.0267 | vs group D: 0.083 | — |
| P value | vs group C: 0.9966 | vs group D: <0.0001 | | |
| | vs group D: 0.0538 | | | |

### 4.2. Duchenne Muscular Dystrophy mdx Model

### 4.2.1. Background

The male mdx mice is the most used animal model for pre-clinical Duchenne muscular dystrophy (DMD) research. (McGreevy et al., 2015)

### 4.2.2. Protocol Overview

Five-week-old mdx mice (Animal Resource Centre; Perth, WA, Australia) were allowed to acclimatise for 1 week to their surroundings, before being randomly allocated to one of four treatment groups or an untreated control group (15 animals per group) as described in the table below. At the start of the study, these groups were matched for mouse body weight.

TABLE V
Mdx model group distribution

| Group (n = 15) | Morning dosing | Afternoon dosing |
|---|---|---|
| Vehicle (Tween-80/Methyl | Vehicle | — |
| cellulose 0.5% (2/98 v/v)) | | |
| Prednisolone (standard of | Prednisolone | — |
| care; 5 mg/kg p.o. qd) | 5 mg/kg | |
| Cpd 1 (120 mg/kg p.o. b.i.d.) | Cpd 1 120 mg/kg | Cpd 1 120 mg/kg |
| Cpd 1 (120 mg/kg p.o. b.i.d.) + | Cpd 1 120 mg/kg + | Cpd 1 120 mg/kg |
| prednisolone (5 mg/kg p.o. qd) | Prednisolone | |
| | 5 mg/kg | |
| No treatment control | — | — |

Following the 1 week period of acclimatisation, i.e. at ˜6 weeks of age, the following baseline measurements were made:
grip strength in accordance to the standard operating procedures for pre-clinical Duchenne muscular dystrophy research as described in the TREAT-NMD guideline (“https://treat-nmd.org/wp-content/uploads/2016/08/MDX-DMD_M.2.2.001.pdf,” n.d.),
body composition using EchoMRI scanner,
spontaneous physical activity and whole-body metabolism using Promethion system to record respiratory gases (O₂ and CO₂), were determined for all treated mice (Cpd 1, prednisolone or Cpd 1+prednisolone, or vehicle).

These measurements were repeated mid-treatment (at ˜10 weeks of age, i.e. 4 weeks of treatment) and at the end of the study (at ˜15 weeks of age, i.e. 9 weeks of treatment) to assess the effects of Cpd 1, prednisolone or Cpd 1+prednisolone on the dystrophic pathology of mdx mice to evaluate the longitudinal, functional assessment of muscle pathology in mdx mice to determine whether drug treatment changes the trajectory of these physiological markers of dystrophic muscle pathology.

The untreated control mdx mice were restrained throughout the study. Following 1-week acclimatisation, this group of mice also have baseline measurements of grip strength and body composition taken, which were repeated at ˜10 weeks and ˜15 weeks of age. Furthermore, these mice were weighed on a regular basis and their urine was collected at ˜6, ˜10 and ˜15 weeks of age.

At ˜11-13 weeks of age (and following 5-7 weeks of treatment), 200 μl of blood was collected from mdx mice treated with Cpd 1 or Cpd 1+prednisolone for pharmacokinetic analysis of serum concentration of Cpd 1. For each of the 2 treatment groups Cpd 1 and Cpd 1+prednisolone, blood samples were taken at the following time points:
−0 h (pre-dose, N=4 mice)
−0.25 h post-dose (N=4 mice),
−2 h post-dose (N=4 mice)
−6 h post-dose (N=3 mice)

To assess the effects of Cpd 1 or Cpd 1+prednisolone on ECM remodelling (for example the proteolytic cleavage of ADAMTS-5 ECM protein substrates such as versican (Stupka et al., 2013)), urine were collected three times—pre, mid and post-treatment at ˜6, ˜10 and ˜15 weeks of age for analysis of matrikines (degraded fragments of extracellular matrix proteins).

Following 9 weeks of treatment, mdx mice (n=3-4 mice per day) were tested for contractile function (hindlimb (tibialis anterior) and diaphragm muscles). The treatment of mice was staggered (n=3-4 mice per treatment group) starting the study each week.

### 4.2.3. Contractile Function Testing

Procedures relating to contractile function testing of tibialis anterior and diaphragm muscles were described below. These were standard procedures for contractile function testing.

To assess the contractile function of tibialis anterior muscles in situ, mice were anaesthetized and the distal portion of the tibialis anterior (TA) muscle and its tendon were exposed.

The tendon was tied with a top and bottom knot using braided surgical thread, then the distal tendon was severed, and the distal portion of the TA muscle was dissected free from surrounding tissue. The sciatic nerve was exposed above the knee joint. The mouse was then secured proximally on the heated platform of the contractile function apparatus. The distal end of the TA were tied firmly to a lever arm attached to an isometric force transducer which was connected to a computer to record force output. Throughout the contractile function testing protocol, warmed physiological saline will be applied to exposed muscle and nerve tissue. Electrical pulses was delivered to the sciatic nerve which produces contraction of the TA muscle, and this contraction was measured by the force transducer and recorded.

Finally, following in situ contractile function testing, the mice were sacrificed. The left and right TA, extensor digitorum longus (EDL), soleus and quadriceps muscles were collected for histological, immunohistochemical, and biochemical analyses.

Blood was also collected for assessment of biomarkers of dystrophic muscle pathology.

The diaphragm and the heart were also collected and the diaphragm, was placed in an organ bath bubbled with carbogen; 5% CO₂ & 95% O₂ for ex vivo contractile function testing. Finally long bones (femur and tibia) and vertebrae were also collected for further analysis.

### 4.2.4. Biomarkers

Matrikines were measured in in urine collected at pre, mid and end of treatment.

Plasma samples were also analysed.

RNA-seq & muscle gene expression were evaluated in the diaphragm and TA muscles samples collected in RNA-later.

### 4.2.5. Skeletal Muscles

Myofibre size and % of centrally nucleated myofibres (a marker of damage and regeneration) were also analysed, including laminin (basal lamina marker) immunohistochemistry and quantitative image analysis.

Muscle progenitor cells and newly regenerated myofibres were also evaluated via desmin immunohistochemistry and quantitative image analysis.

Inflammation markers such as CD68 (a monocyte & pan-macrophage marker) were evaluated by immunohistochemistry and quantitative image analysis.

Fibrosis markers were analysed via histology using Sirius red for collagen and fluorescein isothiocyanate—Triticum vulgaris Lectin (FITC-WGA) for ECM glycoconjugates and quantitative image analysis; hydroxyproline assay for collagen content.

Intramuscular adipocytes were manually counted on Hematoxylin and Eosin-(H&E) stained muscle cross-sections as an initial assessment.

### 4.2.6. Bones

Bone strength was evaluated via 3-point bending on the tibia.

Bone structure was evaluated via μCT analysis of cortical and trabecular bone on the femur.

Growth plate structure was evaluated via Hematoxylin and Eosin-(H&E) staining (femur) and quantitative image analysis. GC treatment has deleterious effects on the growth plate.

Femur histomorphometry was performed on marrow adipose tissue (von Kossa); osteoblasts (ALP); osteoclasts (TRAP).

Vertebral bone structure was evaluated via μCT analysis

### 4.2.7. Results

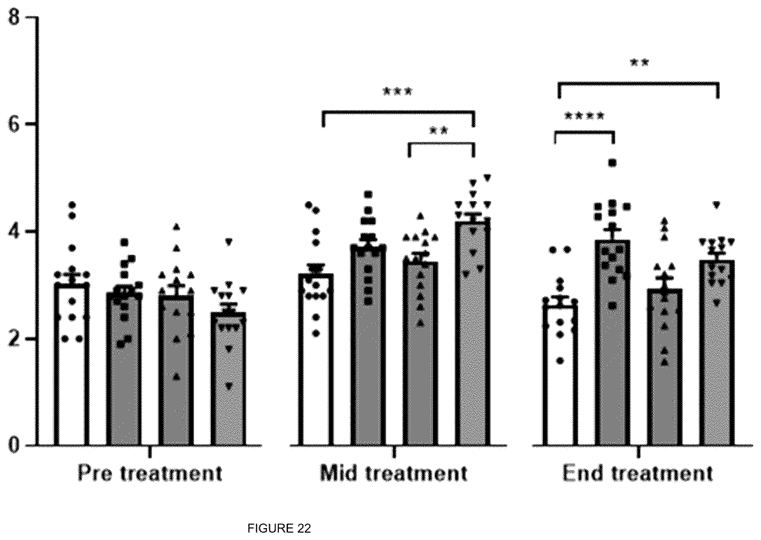

When subjected to the above protocol, Cpd 1 showed a statistically significant improvement in muscle grip strenght compared to the control groups (FIG. 22).

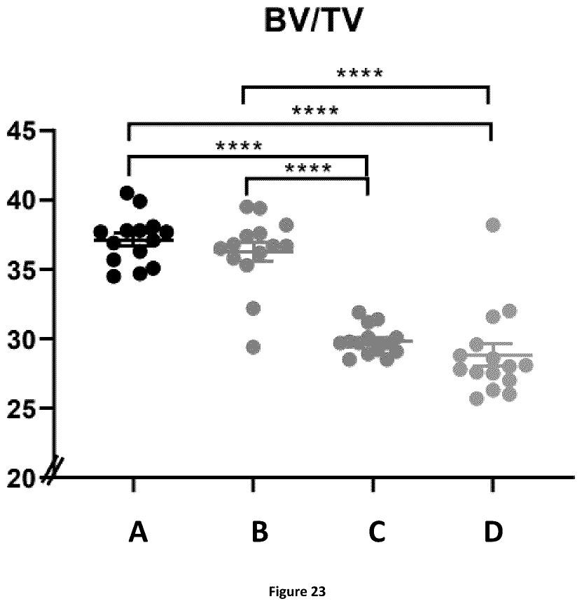

Moreover, a statistically significant retention in bone and tissue volume was observed (FIG. 23), in contrast with the prednisolone treated group, the standard of care in Duchenne dystrophy, which is associated with bone loss. (Novotny et al., 2012)

TABLE VI
Grip strenght results - FIG. 22 (vehicle (filled circles),
Cpd 1 (filled squares), prednisolone (filled upward triangles),
and Cpd 1 + prednisolone (filled downwards triangles))

| | | | | | | | | Cpd 1 + | |
|---|---|---|---|---|---|---|---|---|---|
| | | Vehicle | | Cpd 1 | | prednisolone | | prednisolone | |
| | | Average | Av. | Average | Av. | Average | Av. | Average | Av. |
| Group (n = 15) | | force | g/g | force | g/g | force | g/g | force | g/g |
| time point | | output | BW | output | BW | output | BW | output | BW |
| pre- | Mean | 61.95 | 3.02 | 61.13 | 2.86 | 60.55 | 2.83 | 52.54 | 2.49 |
| treat- | SD | 16.27 | 0.71 | 10.88 | 0.50 | 15.19 | 0.68 | 11.66 | 0.59 |
| ment | SEM | 4.20 | 0.18 | 2.81 | 0.13 | 3.92 | 0.18 | 3.01 | 0.15 |
| mid | Mean | 80.66 | 3.21 | 91.88 | 3.72 | 84.86 | 3.44 | 93.79 | 4.20 |
| treat- | SD | 16.68 | 0.66 | 12.36 | 0.53 | 10.55 | 0.55 | 12.19 | 0.53 |
| ment | SEM | 4.31 | 0.17 | 3.19 | 0.14 | 2.72 | 0.14 | 3.15 | 0.14 |
| post | Mean | 75.28 | 2.63 | 88.24 | 3.86 | 78.84 | 2.93 | 85.92 | 3.57 |
| treat- | SD | 13.43 | 0.54 | 15.21 | 0.69 | 19.26 | 0.75 | 13.74 | 0.55 |
| ment | SEM | 3.47 | 0.14 | 3.93 | 0.18 | 4.97 | 0.19 | 3.55 | 0.14 |

Av g/g BW: average force output/g of body weight
SD: standard deviation
SEM: standard error of the mean

TABLE VII
Bone loss measurements - FIG. 23

| | | | | BV/TV |
|---|---|---|---|---|
| | | Tissue | Bone | (bone volume |
| Group | | volume | volume | fraction) |
| A - vehicle (n = 14) | mean | 2.26 | 0.84 | 37.13 |
| | SD | 0.25 | 0.07 | 1.72 |
| | SEM | 0.07 | 0.02 | 0.46 |
| B - Cpd 1 (n = 14) | mean | 2.21 | 0.80 | 36.09 |
| | SD | 0.17 | 0.06 | 2.57 |
| | SEM | 0.04 | 0.02 | 0.66 |
| C - prednisolone (n = 14) | mean | 2.34 | 0.70 | 29.84 |
| | SD | 0.19 | 0.06 | 1.01 |
| | SEM | 0.05 | 0.02 | 0.27 |
| D - Cpd1 + prednisolone | mean | 2.17 | 0.63 | 28.85 |
| (n = 15) | SD | 0.14 | 0.06 | 3.05 |
| | SEM | 0.04 | 0.02 | 0.79 |

### 4.3. rat CDHFD Model

### 4.3.1. General Overview

The choline-deficient, L-amino acid-defined, high-fat diet (CDHFD) dietary model is a model that develops steatohepatitis, liver fibrosis and hepatocarcinogenesis similar to MCD diet (Santhekadur et al., 2017) and is used to evaluate the compounds of the invention.

### 4.3.2. Animals

At induction, 8 weeks-old male Wistar Han rats (Charles River, France) maintained at 22° C.±2° C. and humidity at 55%±10%, with a 12-hrs dark/light cycle were fed a standard chow diet (A124550KR, Research Diet, USA) or High fat (45% kCal fat) choline deficient diet with 0.1% methionine and 1% cholesterol (CDHF) diet (A16092003, Research Diet, USA) for 12 weeks. All animals have access to filtered tap drinking water.

### 4.3.3. Study

Six weeks after the induction, the animals were either assigned to a control-group or the test-group. Rats were randomly assigned to a treatment group according to their body weight, serum bilirubin and transaminase levels to ensure a homogenous reparation. Test group animals were dosed with the test compound at 50 mg/kg p.o. b.i.d. methyl cellulose 0.5%. The control groups receive a similar volume of vehicle (10 mL/kg), i.e. the standard diet for control group 1 (C1) and the CDHF diet for control groups 2 (C2, 12 weeks) and the CDHF diet+positive control formulated in 0.5% methyl cellulose +98.9% water (Cpd C, 12 weeks).

After sacrifice (week 12), the activity of the test compound on the development of NASH was assessed by plasma alanine aminotransferase (ALT), Alkaline phosphatase (ALP) and aspartate aminotransferase (AST) levels in serum, assessed by Enhanced Liver Fibrosis (ELF) biomarker quantification and in liver by histopathological examination of fibrosis and steatosis (Sirius red, Oil Red O), and lipids levels (triglycerides, Non-Esterified Fatty Acids (NEFA), Cholesterol) content and expression of fibrotic and inflammatory genes.

### 4.3.4. Results

When subjected to this protocol, test compound dosed at 50 mg/kg p.o. b.i.d. in methyl cellulose 0.5% showed a statistically significant reduction of AST (−27%), alpha2 macroglobulin (−63%), procollagen (−48%) and hyaluronan (−65%) levels in serum when compared to the vehicle group. In liver, test compound showed statistically significant reduction of liver fibrosis (−48%).

### 4.4. Graft Versus Host (eGvhD) Lung Model

### 4.4.1. General Overview

In this cGvHD model, fibrosis was induced in BALB/c (H2^{d}) mice by allogeneic transplantation of bone marrow cells and splenocytes from B10.D2 (H2^{d}) donor mice (minor HLA mismatch). The recipient mice develop inflammation-driven dermal and pulmonary fibrosis resembling patients with rapidly progressive diffuse cutaneous systemic sclerosis (Zen et al., 2012).

The treatment was provided only after the onset of first clinical symptoms of sclerodermatous cGvHD.

### 4.4.2. Study Groups

The following groups with each eight mice were used in this study
Syngeneically transplanted, placebo-treated control group: Syngeneic bone marrow and splenocyte transplantation (BALB/c (H2^{d})→BALB/c (H2^{d})). Application of methyl cellulose 0.5% from day 21 to day 56 post transplantation.
Vehicle-treated fibrosis group: Allogeneic bone marrow and splenocyte transplantation (B10.D2 (H2^{d})→BALB/c (H2^{d})). Application of methyl cellulose 0.5% from day 21 to day 56 post transplantation
Control group to assess pretreatment levels of fibrosis induced by allogeneic transplantation: Allogeneic bone marrow and splenocyte transplantation (B10.D2 (H2^{d})→BALB/c (H2^{d})). Sacrifice at day 21, before treatment was initiated in the other groups.
Treatment group: Allogeneic bone marrow and splenocyte transplantation (B10.D2 (H2^{d})→BALB/c (H2^{d})). Application of a test compound of the invention from day 21 to day 56 post transplantation.
Positive control group: Allogeneic bone marrow and splenocyte transplantation (B10.D2 (H2^{d})→BALB/c (H2^{d})). Application of 50 mg/kg qd nintedanib from day 21 to day 56 post transplantation.

### 4.4.3. Steady State PK

On D20, for the groups receiving test compounds, blood was collected from the tail vein from 2 animals per timepoint, at the following timepoints: pre-dose, 1, 3 and 6 h with anticoagulant Li-heparin.

The blood samples were kept on ice and centrifuged at approx. 3500×g, for 10 min at +4° C., within 1 h after blood sampling; plasma was transferred in polypropylene tubes and stored at −20° C.

### 4.4.4. Sampling and Analysis

Animals were sacrificed 2 h post last dose, and samples of skin (3 mm punch biopsies), lung, spleen and blood were collected for histology and gene expression analysis.

### 4.4.5. Main Readouts

The anti-fibrotic effects on skin were analysed by determination of dermal thickness, quantification of lesional collagen and staining for myofibroblasts.

In case of positive effects on skin fibrosis, effects on pulmonary fibrosis were analysed by Ashcroft scoring, hydroxyproline content, and quantification of the collagen covered area using Sirius red staining

### 4.4.6. Analysis

Based on individual animal raw data, the means for each group were determined and percent change from disease controls was calculated. Treatment groups were compared to disease controls using a one-way analysis of variance (1-way ANOVA) with a Dunnett's post-hoc analysis for measured (parametric) data or a Kruskal-Wallis test with a Dunn's post-hoc analysis for scored (non-parametric) data.

### 4.4. 7. Results

When subjected to this protocol, Cpd 1 dosed at 120 mg/kg p.o. b.i.d. in Tween 80/methyl cellulose 0.5% (2/98) showed a statistically non-significant reduction of dermal thickness, but a statistically significant reduction of myofibroblast count (−35%) and Hydroxyproline content in skin (−8.3%).

In lung, Cpd 1 showed a statistically significant decrease of Ashcroft score (−1,3 fold), and collagen-covered lung area (−1,2 fold) compared to the vehicle group.

### FINAL REMARKS

It will be appreciated by those skilled in the art that the foregoing descriptions are exemplary and explanatory in nature and intended to illustrate the invention and its preferred embodiments. Through routine experimentation, an artisan will recognize apparent modifications and variations that may be made without departing from the spirit of the invention. All such modifications coming within the scope of the appended claims are intended to be included therein. Thus, the invention is intended to be defined not by the above description, but by the following claims and their equivalents.

### REFERENCES

Abbaszade, I., Liu, R.-Q., Yang, F., Rosenfeld, S. A., Ross, O. H., Link, J. R., Ellis, D. M., Tortorella, M. D., Pratta, M. A., Hollis, J. M., Wynn, R., Duke, J. L., George, H. J., Hillman, M. C., Murphy, K., Wiswall, B. H., Copeland, R. A., Decicco, C. P., Bruckner, R., Nagase, H., Itoh, Y., Newton, R. C., Magolda, R. L., Trzaskos, J. M., Hollis, G. F., Arner, E. C., Burn, T. C., 1999. Cloning and Characterization of ADAMTS11, an Aggrecanase from the ADAMTS Family. J. Biol. Chem. 274, 23443-23450.
Addinsall, A., Forgan, L., McRae, N., Kelly, R., McDonald, P., McNeil, B., McCulloch, D., Stupka, N., 2020. Treatment of Dystrophic mdx Mice with an ADAMTS-5 Specific Monoclonal Antibody Increases the Ex Vivo Strength of Isolated Fast Twitch Hindlimb Muscles. Biomolecules 10, 416. https://doi.org/10.3390/biom10030416
Bauters, D., Bedossa, P., Lijnen, H. R., Hemmeryckx, B., 2018. Functional role of ADAMTS5 in adiposity and metabolic health. PLOS ONE 13, e0190595. https://doi.org/10.1371/journal.pone.0190595
Bauters, D., Spincemaille, P., Geys, L., Cassiman, D., Vermeersch, P., Bedossa, P., Scroyen, I., Lijnen, H. R., 2016. ADAMTS5 deficiency protects against non-alcoholic steatohepatitis in obesity. Liver Int. 36, 1848-1859. https://doi.org/10.1111/liv.13181
Botter, S. M., Glasson, S. S., Hopkins, B., Clockaerts, S., Weinans, H., van Leeuwen, J. P. T. M., van Osch, G. J. V. M., 2009. ADAMTS5−/−mice have less subchondral bone changes after induction of osteoarthritis through surgical instability: implications for a link between cartilage and subchondral bone changes. Osteoarthritis Cartilage 17, 636-645. https://doi.org/10.1016/j.joca.2008.09.018
Chiusaroli, R., Visintin, M., Caselli, G., Rovati, L. C., 2013. Anti-Adamts-5 Antibody, Derivatives and Uses Thereof. WO2013153189 (A1).
Chockalingam, P. S., Sun, W., Rivera-Bermudez, M. A., Zeng, W., Dufield, D. R., Larsson, S., Lohmander, L. S., Flannery, C. R., Glasson, S. S., Georgiadis, K. E., Morris, E. A., 2011. Elevated aggrecanase activity in a rat model of joint injury is attenuated by an aggrecanase specific inhibitor. Osteoarthritis Cartilage 19, 315-323. https://doi.org/10.1016/j.joca.2010.12.004
Collins, I., Wann, A. K. T., 2020. Regulation of the Extracellular Matrix by Ciliary Machinery. Cells 9, 278. https://doi.org/10.3390/cells9020278
Glasson, S. S., Askew, R., Sheppard, B., Canto, B., Blanchet, T., Ma, H.-L., Flannery, C. R., Peluso, D., Kanki, K., Yang, Z., Majumdar, M. K., Morris, E. A., 2005. Deletion of active ADAMTS5 prevents cartilage degradation in a murine model of osteoarthritis. Nature 434, 644-648. https://doi.org/10.1038/nature03369
Hilfiker, R., Blatter, F., Raumer, M. von, 2006. Relevance of Solid-state Properties for Pharmaceutical Products, in: Hilfiker, R. (Ed.), Polymorphism. Wiley-VCH Verlag GmbH & Co. KGaA, pp. 1-19. https://insights.envigo.com/hubfs/resources/data-sheets/2018-datasheet-0915 n.d. https://treat-nmd.org/wp-content/uploads/2016/08/MDX-DMD_M.2.2.001.pcif, n.d.
Larsson, S., Lohmander, L. S., Struglics, A., 2014. An ARGS-aggrecan assay for analysis in blood and synovial fluid. Osteoarthritis Cartilage 22, 242-249. https://doi.org/10.1016/j.joca.2013.12.010
Lipinski, C. A., Lombardo, F., Dominy, B. W., Feeney, P. J., 2001. Experimental and computational approaches to estimate solubility and permeability in drug discovery and development settings. Adv. Drug Deliv. Rev. 46, 3-26. https://doi.org/10.1016/50169-409X(00)00129-0
Little, C. B., Meeker, C. T., Golub, S. B., Lawlor, K. E., Farmer, P. J., Smith, S. M., Fosang, A. J., 2007. Blocking aggrecanase cleavage in the aggrecan interglobular domain abrogates cartilage erosion and promotes cartilage repair. J. Clin. Invest. 117, 1627-1636. https://doi.org/10.1172/JCI30765
Malfait, A. M., Ritchie, J., Gil, A. S., Austin, J.-S., Hartke, J., Qin, W., Tortorella, M. D., Mogil, J. S., 2010. ADAMTS-5 deficient mice do not develop mechanical allodynia associated with osteoarthritis following medial meniscal destabilization. Osteoarthritis Cartilage 18, 572-580. https://doi.org/10.1016/j.joca.2009.11.013
McGreevy, J. W., Hakim, C. H., McIntosh, M. A., Duan, D., 2015. Animal models of Duchenne muscular dystrophy: from basic mechanisms to gene therapy. Dis. Model. Mech. 8, 195-213. https://doi.org/10.1242/dmm.018424
McMahon, M., Ye, S., Izzard, L., Dlugolenski, D., Tripp, R. A., Bean, A. G. D., McCulloch, D. R., Stambas, J., 2016. ADAMTS5 Is a Critical Regulator of Virus-Specific T Cell Immunity. PLOS Biol. 14, e1002580. https://doi.org/10.1371/journal.pbio.1002580
Novotny, S. A., Warren, G. L., Lin, A. S., Guldberg, R. E., Baltgalvis, K. A., Lowe, D. A., 2012. Prednisolone treatment and restricted physical activity further compromise bone of mdx mice. J. Musculoskelet. Neuronal Interact. 12, 16-23.
Pardo, A., Selman, M., Kaminski, N., 2008. Approaching the degradome in idiopathic pulmonary fibrosis. Int. J. Biochem. Cell Biol., Directed Issue: Proteases and Antiproteases in Development, Homeostasis and Disease 40, 1141-1155. https://doi.org/10.1016/j.bioce1.2007.11.020
Santhekadur, P. K., Kumar, D. P., Sanyal, A. J., 2017. Preclinical Models of Nonalcoholic Fatty Liver Disease. J. Hepatol. https://doi.org/10.1016/j.jhep.2017.10.031
Stahl, P. H., Wermuth, C. G., International Union of Pure and Applied Chemistry, 2011. Handbook of pharmaceutical salts: properties, selection, and use. VHCA; Weinheim: Wiley-VCH, Zürich.
Stanton, H., Rogerson, F. M., East, C. J., Golub, S. B., Lawlor, K. E., Meeker, C. T., Little, C. B., Last, K., Farmer, P. J., Campbell, I. K., Fourie, A. M., Fosang, A. J., 2005. ADAMTS5 is the major aggrecanase in mouse cartilage in vivo and in vitro. Nature 434, 648-652. https://doi.org/10.1038/nature03417
Stupka, N., Kintakas, C., White, J. D., Fraser, F. W., Hanciu, M., Aramaki-Hattori, N., Martin, S., Coles, C., Collier, F., Ward, A. C., Apte, S. S., McCulloch, D. R., 2013. Versican Processing by a Disintegrin-like and Metalloproteinase Domain with Thrombospondin-1 Repeats Proteinases-5 and -15 Facilitates Myoblast Fusion*. J. Biol. Chem. 288, 1907-1917. https://doi.org/10.1074/jbc.M112.429647
Taylor, S., Whitfield, M., Barratt, J., Didangelos, A., 2020. The Metalloproteinase ADAMTS5 Is Expressed by Interstitial Inflammatory Cells in IgA Nephropathy and Is Proteolytically Active on the Kidney Matrix. J. Immunol. 205, 2243-2254. https://doi.org/10.4049/jimmunol.2000448
Zerr, P., Distler, A., Palumbo-Zerr, K., Tomcik, M., Vollath, S., Dees, C., Egberts, F., Tinazzi, I., Del Galdo, F., Distler, O., Schett, G., Spriewald, B. M., Distler, J. H. W., 2012. Combined Inhibition of c-Abl and PDGF Receptors for Prevention and Treatment of Murine Sclerodermatous Chronic Graft-versus-Host Disease. Am. J. Pathol. 181, 1672-1680. https://doi.org/10.1016/j.ajpath.2012.07.017

1. A solid form of a compound according to Formula I: 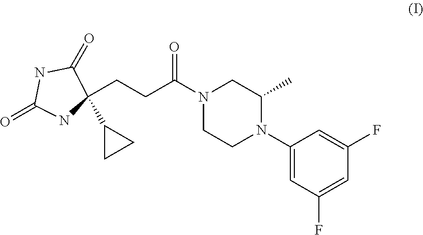 [SMILES: C[C@H]1CN(C(=O)CC[C@@]2(C3CC3)NC(=O)NC2=O)CCN1c1cc(F)cc(F)c1]
or a pharmaceutically acceptable solvate thereof.

2. The solid form according to claim 1 characterized by an X-ray powder diffraction pattern comprising peaks at 6.2, 12.5, 15.7, 19.1, 25.2, 26.4±0.2° 2θ using Cu Kα radiation.

3. The solid form according to claim 1 characterized by an X-ray powder diffraction pattern comprising peaks at 6.2, 12.5, 14.1, 15.7, 19.1, 21.4, 22.4, 25.2, 26.4±0.2° 2θ using Cu Kα radiation

4. The solid form according to claim 1 characterized by an X-ray powder diffraction pattern comprising peaks at 6.2, 12.5, 14.1, 14.6, 15.6, 15.7, 17.8, 18.0, 18.8, 19.1, 19.7, 20.8, 21.4, 22.4, 25.2, 26.4, 28.9, 29.0±0.2° 2θ using Cu Kα radiation.

5. The solid form according to any one of claims 1 to 3 having an X-ray powder diffraction profile substantially as shown in FIG. 1.

6. The solid form according to any one of claims 1 to 5 having an endothermic transition at about 80° C., as measured by differential scanning calorimetry.

7. The solid form according to any one of claims 1, characterized by an X-ray powder diffraction pattern comprising peaks at 10.3, 15.3, 15.7, 16.7, 18.6±0.2° 2θ using Cu Kα radiation.

8. The solid form of claim 7 further characterized by an X-ray powder diffraction pattern comprising peaks at 16.7, 24.5, 30.4±0.2° 2θ using Cu Kα radiation.

9. The solid form of claim 7 or 8 further characterized by an X-ray powder diffraction pattern comprising peaks at 8.5, 12.6, 13.2, 13.6, 14.7, 18.3, 20.3, 20.8, 22.9, 27.2±0.2° 2θ using Cu Kα radiation.

10. The solid form according to any one of claims 1 and 7 to 9 having an X-ray powder diffraction pattern substantially as shown in FIG. 2.

11. The solid form according to any one of claims 1 and 7 to 10 having an endothermic transition at about 159° C., as measured by differential scanning calorimetry.

12. A process for the preparation of a solid form according to any one of claims 2-6 comprising:
a) Admixing amorphous (5S)-cyclopropyl-5-[3- [(3S)-4-(3,5-difluorophenyl)-3-methyl-piperazin-1-yl]-3-oxo-propyl]imidazolidine-2,4-dione and isopropyl alcohol,
b) Heating the mixture to about 40° C.,
c) Adding a first batch of water,
d) Cooling the mixture to about 5° C. at a rate of about 0.3° C./min
e) Adding a second batch of water,
f) Filtrating the mixture,
g) Washing the cake with cooled water,
h) Drying the solid.

13. A process for the preparation of a solid form according to any one of claims 7-11 comprising:
a) Admixing amorphous (5S)-cyclopropyl-5-[3-[(3S)-4-(3,5-difluorophenyl)-3-methyl-piperazin-1-yl]-3-oxo-propyl]imidazolidine-2,4-dione and isopropyl alcohol,
b) Heating the mixture to about 40° C.,
c) Cooling the mixture to about 25° C. at a rate of about 0.1° C./min,
d) Adding methyl cyclohexane,
e) Cooling the mixture to about 5° C. at a rate of about 0.1° C./min,
f) Filtrating the mixture,
g) Washing the cake with cooled methlyl cyclohexane,
h) Drying the solid.

14. A process for the preparation of a solid form according to any one of claims 7-11 comprising:
a) Admixing amorphous (5S)-cyclopropyl-5-[3-[(3S)-4-(3,5-difluorophenyl)-3-methyl-piperazin-1-yl]-3-oxo-propyl]imidazolidine-2,4-dione and methyl isobutyl ketone,
b) heating the reaction to about 60° C.,
c) Adding diisopropyl ether,
d) Cooling the mixture to about 0° C. in steps of 10° C. at a rate of about 0.3° C./min with contact times of at least 60 min between each steps,
e) Filtrating the mixture,
f) Washing the cake with cooled diisopropyl ether,
g) Drying the solid.

15. A pharmaceutical composition, comprising a compound or pharmaceutically acceptable salt thereof accord in to any one of claims 1-11, and a pharmaceutically acceptable carrier.

16. A compound or a pharmaceutically acceptable salt thereof according to any one of claims 1-11, or a pharmaceutical composition according to claim 15, for use in the treatment of prophylaxis and/or treatment of inflammatory conditions, muscular disease, fibrotic diseases, viral infection, and/or diseases involving degradation of cartilage and/or disruption of cartilage homeostasis.
